(19) United States
(12) Patent Application Publication
(10) Pub. No.: US 2023/0416678 A1
(43) Pub. Date: Dec. 28, 2023

(54) CELL CULTURE MEDIUM

(71) Applicant: Auckland UniServices Limited, Auckland (NZ)

(72) Inventors: Peter Roderick Dunbar, Auckland (NZ); Vaughan John Feisst, Auckland (NZ); Inken Dorothea Kelch, Auckland (NZ)

(21) Appl. No.: 18/361,233

(22) Filed: Jul. 28, 2023

Related U.S. Application Data
(63) Continuation of application No. 16/648,414, filed on Mar. 18, 2020.

(30) Foreign Application Priority Data
Sep. 30, 2017 (GB) 1715930.2

(51) Int. Cl. C12N 5/071; A61F 2/10; A61K 35/36; A61L 27/38; A61L 27/60

(52) U.S. Cl. CPC C12N 5/0629; A61F 2/105; A61K 35/36; A61L 27/3813; A61L 27/3886; A61L 27/60; C12N 2501/01; C12N 2501/117; C12N 2501/727; C12N 2501/998; C12N 2501/999

(57) ABSTRACT

The present invention relates to a growth medium, which does not include cholera toxin, for culturing cells, such as epithelial cells, in particular keratinocytes, use of the medium to grow cells, (for example epithelial cells, such as keratinocytes) and kits comprising the medium. Also provided is a method of generating human full thickness skin, fully human epidermis or fully human dermis involving culturing keratinocytes and feeder cells in said cell culture medium.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application is a continuation of U.S. application Ser. No. 16/648,414, filed Mar. 18, 2020, which is a National Stage Entry of PCT/NZ2018/050132, filed Sep. 28, 2018, which claims priority to GB 1715930.2 filed on Sep. 30, 2017, the entire contents of which are incorporated herein by reference.

### BACKGROUND OF THE INVENTION

Engineered tissue comprising an epidermis, for example grown in vitro, has a wide range of uses. Such tissue is used in skin grafts for patients with burns or chronic wounds or in the development and testing of pharmaceutical, cosmetic and other topical products. Keratinocytes which differentiate to form epidermis need to be co-cultured with fibroblastic feeder cells.

In the production of skin grafts, keratinocytes are commonly grown with Green's medium and irradiated xenogeneic mouse embryonic feeder cells (MEFs). Unirradiated murine feeder cells outgrow the keratinocytes and swamp the culture. Therefore, the feeder cells are irradiated to stop or slow their replication.

Green's medium is a complex mix of ingredients including:

Dulbecco's Modified Eagles Medium (DMEM) which contains calcium chloride, ferric nitrate, magnesium sulfate, potassium chloride, sodium bicarbonate, sodium chloride, sodium phosphate moonbasic (anhydrous), L-arginine·HCl, L-cysteine·HCl, glycine, L-histidine·HCl·H2O, L-isoleucine, L-leucine, L-lysine HCl, L-methionine, L-tryptophan, L-tyrosine·2Na·H2O, L-valine, choline chloride, folic acid, myo-inositol, niacinamide, D-pantothenicc acid, pyridoxal·HCl, pyridoxine, riboflavin, thiamine·HCl, D-glucose, phenol red sodium, pyruvic acid;

Ham F12 medium which comprises calcium chloride, cupric sulfate·5H2O, ferrous sulfate·7H2O, magnesium chloride, potassium chloride, sodium bicarbonate, sodium chloride, sodium phosphate dibasic (anhydrous), zinc sulfate·7H2O, L-alanine, L-arginine·HCl, L-asparagine·H2O, L-aspartic acid, L-cysteine·HCl·H2O, L-isoleucine, L-leucine, L-lysine·HCl, L-methionine, L-phenylalanine, L-proline, L-serine, L-threonine, L-tryptophan, L-tryptophan, L-tyrosine·2Na·H2O, L-valine, D-biotin, choline chloride, folic acid, myo-inositol, niacinamide, D-pantothenic acid, pyridoxine HCl, riboflavin, thiamine·HCl, vitamin B12, D-glucose, hypoxanthine, linoleic acid, phenol red·Na, thiotic acid, and thymidine; fetal bovine serum; cholera toxin; recombinant human insulin; hydrocortisone; L′ glutamine, EGF, apotransferrin adenine and 3,3,5,-tri-idothyronine and two or three antibiotics.

Regulatory approval of GMP manufacturing processes, generally requires some validation and scientific support for the use supplementary components, like media, unless the material is categorised as a “gras reagent” (generally regarded as safe reagent).

Green's medium has never been optimised for the growth of epithelial cells, such as keratinocytes. Green's medium components were investigated by the present inventors to identify which have a minimal impact (and therefore are superfluous), or may even have a detrimental effect on keratinocyte growth. However, because of the complicated nature of the medium it is difficult to establish what components are essential and which are inessential.

Okada et al J. Inves. Dermatol. 1982 July 79(1) 42-7 discloses that cholera toxin accelerates the growth of keratinocytes. Clearly overall processing time to generate skin product is an important factor of process efficiency and viability and thus to date cholera toxin was thought to be essential aspect of the media.

However, the presence of cholera toxin in the medium is a potential problem, because it is a potent immunomodulator, and it exhibits both mucosal and systemic adjuvant activities. Adjuvant activity is appropriate in vaccines because it increases the immune response to the antigen in the vaccine. However, in skin grafts increased immune responses may be undesirable because these responses may increase the risk of graft rejection.

In addition, in some instances choleratoxin is grown employing bovine brain tissue, which carries with it a risk of contamination by prions.

The use of xenogenic feeder cells means the skin samples grown in vitro are not fully human. Residual non-human components/proteins in the engineered tissue may generate intolerance to the graft and generate immune responses that contribute to graft rejection. Exposure of therapeutic products to xenogeneic cells also risks transmission of infectious agents from non-human hosts.

At the present time, no-one in the field makes fully human skin products, for example comprising a fully human epidermis, in particular fully differentiated full thickness, fully human skin products, in particular without the use of cholera toxin in the media.

It would also be useful to have a cell culture medium that allows the culture of epithelial cells, such as keratinocytes with unirradiated human fibroblasts, for example in the preparation of engineered skin products comprising a fully human epidermis, such as full-thickness fully human skin. Hence, there is a need for an improved medium for growing keratinocytes, in particular a simplified medium, which is robust and reproducible and suitable for use in GMP manufacturing.

### SUMMARY OF THE INVENTION

The invention is summarised in the following paragraphs:

A cell culture medium comprising or consisting of components: DMEM, Ham's F12, serum, one or more antibiotic(s) and/or antimycotic(s), keratinocyte growth factor (KGF) &/or epidermal growth factor and optionally a keratinocyte growth accelerator factor wherein the factor is other than cholera toxin.

A cell culture medium according to paragraph 1, wherein the DMEM is high glucose.

A cell culture medium according to paragraph 1 or 2, wherein the serum is mammalian, for foetal bovine serum.

A cell culture medium according to any one of paragraphs 1 to 3, wherein the serum is present at a concentration in the range 5 to 15%, such as 5, 6, 7, 8, 9, 10, 11, 12, 13, 14 or 15%, in particular 10%.

A cell culture medium according to any one of paragraphs 1 to 4, wherein at least one antibiotic is a component (for example 1, 2, or 3 antibiotics).

A cell culture medium according to paragraph 5, wherein the antibiotic is independently selected from penicillin, streptomycin, ampicillin, amoxicillin, carbenicillin, cefotaxime, gentamicin, kanamycin, neomycin, polymyxin, blasticidin, geneticin, Hygromycin B, Mycophenolic acid, puromycin, zeocin and combinations of two or more of the same.

A cell culture medium according to paragraph 5 or 6, wherein the antibiotic is gentamicin,

A cell culture medium according to any one of paragraphs 5 to 7, wherein the antibiotic is penicillin.

A cell culture according to any one of paragraphs 5 to 8, wherein the antibiotic is streptomycin.

A cell culture medium according to any one of paragraphs 1 to 9, wherein an antimycotic is a component.

A cell culture medium according to paragraph 10, wherein the antimycotic is selected from amphotericin B, nystatin, actinomycin D, fosmidomycin, mycophenolic acid and combinations of two or more the same.

A cell culture medium according to paragraph 11, wherein the antimycotic is amphotericin B, for example at a concentration of in the range 0.500 to 0.750 μg/ml, such as 0.625 μg/ml.

A cell culture medium according to any one of paragraphs 1 to 12, wherein the ratio of DMEM:Ham's F12 is 2.5 to 3.5:1.5 to 0.5, such as 3:1.

A cell culture medium according to any one of paragraphs 1 to 13, wherein the KGF has a concentration in the range 10 ng/ml to 30 ng/ml, such as 20 ng/ml.

A cell culture medium according to any one of paragraphs 1 to 14, wherein the keratinocyte growth accelerator factor is a ROCK inhibitor, such as a small molecule ROCK inhibitor.

A cell culture medium according to paragraph 15, wherein the ROCK inhibitor is selected from the group consisting of: SB772077B, Y-27632, Fasudil, Ripasudil, Y39983, Wf-536, SLx-2119, an azabenimidazole-aminofurazan, DE-104, H-1152, ROKα inhibitor, XD-4000, HMN-1152, 4-(1-aminoalkyl)-N-(4-pyridyl)cyclohexane-carboxamide, rhostatin, BA-210, BA-207, BA-215, BA-285, BA-1037, Ki-23095, VAS-012, RKI-1447, GSK429286A, Y-30141, HA-100, H-7, iso H-7, H-89, HA-1004, HA-1077, H-8, H-9, KN-62, GSK269962, quinazoline and combinations of two or more of the same.

A cell culture medium according to paragraph 16, wherein the ROCK inhibitor is SB772077B, Y-27632, or a combination of both, in particular SB772077B.

A cell culture medium according to any one of paragraphs 15 to 17 wherein the ROCK inhibitor concentration is in the range 0.1 to 100 μM, for example 0.2 to 50 μM or 0.3 to 25 μM or 0.1 to 10 μM or 0.1 to 0.95 μM, such as 0.15, 0.2, 0.25, 0.3, 0.35, 0.4, 0.45, 0.5, 0.55, 0.6, 0.65, 0.7, 0.75, 0.8, 0.85, 0.9 or 0.95 μM, in particular 0.4 μM.

An in vitro method of generating a human full thickness skin, a fully human epidermis or a fully human dermis (i.e. comprising a fully human epidermis) comprising a first culturing step wherein human keratinocytes and feeder cells are cultured in cell culture media according to any one of paragraphs 1 to 18.

An in vitro method according to paragraph 19, wherein the feeder cells are fibroblasts.

An in vitro method according to paragraph 20, wherein the fibroblasts are fully human.

An in vitro method according to any one of paragraphs 19 to 21, wherein the human keratinocytes are not cultured in the presence of xenogeneic feeder cells.

An in vitro method according to any one of paragraphs 19 to 22, wherein the human fibroblast cells are not irradiated.

An in vitro method according to any one of paragraphs 19 to 23, wherein the human fibroblast cells are sex matched to the keratinocytes (and or a patient).

An in vitro method according to any one of paragraphs 19 to 24, wherein the human fibroblast cells are HLA matched to the keratinocytes (and or a patient).

An in vitro method according to any one of paragraphs 19 to 25, wherein the human fibroblasts and keratinocytes are from the same donor.

An in vitro method according to any one of paragraphs 19 to 26, wherein the human fibroblasts are allogeneic to a human patient.

An in vitro method according to any one of paragraphs 19 to 27, wherein the human keratinocytes are allogeneic to a human patient.

An in vitro method according to any one of paragraphs 19 to 23 or 25, wherein the human fibroblasts are autologous to a human patient.

An in vitro method according to any one of paragraphs 13 to 27 or 29, wherein the human keratinocytes are autologous to a human patient.

An in vitro method according to any one of paragraphs 19 to 30, wherein the method comprises second culture step with a first culturing phase wherein the keratinocytes are not in contact with a gas permeable membrane (interface) and are not at the liquid interface.

An in vitro method according to paragraph 31, wherein the keratinocytes in the second step are cultured, for a second phase in contact with a gas permeable membrane (interface), for example where the keratinocytes are in contact with the gas permeable membrane (following the period of culturing without contacting a gas permeable layer).

An in vitro method according to any one of paragraphs 19 to 32, wherein the keratinocytes are deposited on a substrate, for example a matrix, for example a spun matrix or mesh.

An in vitro method according to paragraph 33, wherein the substrate (such as a matrix) comprises a biocompatible biodegradable polymer.

An in vitro method according to paragraph 33 or 34, wherein the substrate is prepared by electrospinning.

An in vitro method according to paragraph 30, wherein electrospun fibres from said electrospinning are about 0.3 μm to about 5 μm in diameter or 2 to 5 μm in diameter, such as 1, 1.5, 2, 2.5, 3, 3.5, 4, or 4.5 μm.

An in vitro method according to paragraph 35 or 36, wherein the substrate is prepared from fibres spun from a polymer is selected from the group consisting of PLGA, PLA, PCL, PHBV, PDO, PGA, PLCL, PLLA-DLA, PEUU, cellulose-acetate, PEG-b-PLA, EVOH, PVA, PEO, PVP, blended PLA/PCL, gelatin-PVA, PCT/collagen, sodium aliginate/PEO, chitosan/PEO, chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan, PDLA/HA, PLLA/HA, gelatin/HA, gelatin/siloxane, PLLA/MWNTs/HA, PLGA/HA, dioxanone linear homopolymer (such as 100 dioxanone linear homopolymer) and combinations of two or more of the same.

An in vitro method according to paragraph 37, wherein the polymer is poly(lactic-co-glycolic acid) (PLGA).

An in vitro method according to paragraphs 33 to 38, wherein the concentration of biocompatible biodegradable polymer is 10 to 40% w/v, for example 26% to 40% w/v, for example 11, 12, 13, 14, 15, 16, 17, 18, 19, 20, 21, 22, 23, 24, 25, 26, 27, 28, 29, 30, 31, 32, 33, 34, 35, 36, 37, 38 or 39% w/v, in particular 15, 16, 17, 18, 19, 20 or 21% w/v.

An in vitro method according to any one of paragraphs 33 to 39, wherein the substrate has a thickness of 10 to 100 μm.

An in vitro method according to any one of paragraphs 33 to 40, wherein substrate is three dimensional, and is substantially planar, with two faces.

An in vitro method according to any one of paragraphs 41, wherein at least one face of the substrate is micropatterned, undulating and/or dimpled.

An in vitro method according to claim any one of paragraphs 33 to 42, wherein the substrate is coated with protein, polypeptide or peptide to assist the keratinocytes adhering to the substrate and/or stratification of the cells.

An in vitro method according to paragraph 43, wherein the protein is selected from an extracellular matrix protein (such as collagen, laminin and other extracellular matrix proteins) or peptide thereof, for example a synthetic peptide.

An in vitro method according to paragraph 44, wherein the extracellular matrix protein is selected from the group consisting of collagen IV, collagen I, laminin and fibronectin, or a combination thereof, such as collagen IV.

An in vitro method according to any one of paragraphs 33 to 45, wherein the substrate is located in a culture device comprising a gas permeable membrane, for example such that one face can be orientated to be in contact with the gas permeable layer or said one face is orientate not in contact with (removed from) said gas permeable layer.

An in vitro method according to paragraph 46, wherein the substrate is moveable, for example by rotation or sliding, between the position where one face is contact with the gas permeable lay and the position where said one face is not in contact with (removed from) said gas permeable layer.

An in vitro method according to any one of paragraphs 19 to 47, further comprising the pre-step of digesting a sample comprising human keratinocytes and human fibroblast feeder cells, such as a skin sample, with a protease digest.

An in vitro method according to paragraph 48, wherein protease is selected from dispase, trypsin and combinations thereof.

An in vitro method according to paragraph 49, wherein dispase is employed (for example to digest the epidermis).

An in vitro method according to any one of paragraphs 48 to 50, wherein trypsin is employed (for example to digest the dermis).

An in vitro method according to any one of paragraphs 48 to 51, wherein the digest is performed in the presence of collagenase.

An in vitro method according to any one of paragraphs 48 to 52, wherein the digest is performed without separating the dermis and epidermis (referred to herein as a whole cell digest).

A fully skin product comprising a human epidermis cultured by the method of any one of paragraphs 19 to 53

A fully human skin product according to paragraph 54, which comprises a differentiated dermis and epidermis.

A fully human skin product according to paragraph 54 or 55 for use in treatment.

A fully human epidermis according to paragraph 56, wherein the treatment is for a condition or disease selected from the group consisting of: tissue damage, for example cuts, lacerations, abrasions (such as excoriation), shearing force damage, bites (including animal bites such as dog bites and insect bites); skin regeneration (for example with nerves & organelles); wound healing, for example promoting/enhancing wound healing, including erosions, ulcers (such as diabetic ulcers) wounds from leprosy, wounds from dystrophic epidermolysis, wounds from hidradentitis suppurativa, wounds from mucous membrane pemphigoid, wounds from pemphigoid, wounds from perphigus vulgaris, wounds from pyoderma gangrenosum, wounds from shingles; burn healing including radiation burns, sunburn, chemical burns (such as acid burns and alkali burns), a thermal burn; skin regeneration and repair, for example atrophy, or after excision of tissue, such as where the excision is: cancerous cells skin cells (including melanoma, basal cell carcinoma, squamous cell carcinoma (such as Bowen's disease), extra-mammary Paget's disease), breast cancer, tissue with Darrier's disease, a cyst, a wart (including plantar warts), necrotizing fascilitis (such as methicillin-resistant Staphylococcus aureus, necrotic tissue (such as gangrene), tissue exhibiting Hailey-Hailey disease, tissue exhibiting blisters associated with pemphigus vulgaris; epidermolysis bullosa; enhance skin quality (for example to treat ichthyosis) or appearance; prevention or remediation of skin disorders, for example an abscess, dermatitis (including contact dermatitis), atopic dermatitis, acne, actinic keratosis, rosacea, a carbuncle, eczema, psoriasis, cellulitis, kertosis, pilaris, melasma, impetigo or a fissure; diminishment or abolishment of scar tissues (for example to treat keloids); breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring. In one embodiment the condition or disease is selected from: tissue damage; skin regeneration with nerves & organelles; wound healing, for example promoting/enhancing wound healing, including ulcers such as diabetic ulcers; burn healing; skin regeneration and repair; epidermolysis bulosa; enhance skin quality or appearance; prevention or remediation of skin disorders; diminishment or abolishment of scar tissues; breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring.

A method of treatment comprising suturing a fully human epidermis according to paragraph 54 or 55 to a patient in need thereof, for example for the treatment of a condition or disease selected from the group consisting of: tissue damage, for example cuts, lacerations, abrasions (such as excoriation), shearing force damage, bites (including animal bites such as dog bites and insect bites); skin regeneration (for example with nerves & organelles); wound healing, for example promoting/enhancing wound healing, including erosions, ulcers (such as diabetic ulcers) wounds from leprosy, wounds from dystrophic epidermolysis, wounds from hidradentitis suppurativa, wounds from mucous membrane pemphigoid, wounds from pemphigoid, wounds from perphigus vulgaris, wounds from pyoderma gangrenosum, wounds from shingles; burn healing including radiation burns, sunburn, chemical burns (such as acid burns and alkali burns), a thermal burn; skin regeneration and repair, for example atrophy, or after excision of tissue, such as where the excision is: cancerous cells skin cells (including melanoma, basal cell carcinoma, squamous cell carcinoma (such as Bowen's disease), extra-mammary Paget's disease), breast cancer, tissue with Darrier's disease, a cyst, a wart (including plantar warts), necrotizing fascilitis (such as methicillin-resistant Staphylococcus aureus, necrotic tissue (such as gangrene), tissue exhibiting Hailey-Hailey disease, tissue exhibiting blisters associated with pemphigus vulgaris; epidermolysis bullosa; enhance skin quality (for example to treat ichthyosis) or appearance; prevention or remediation of skin disorders, for example an abscess, dermatitis (including contact dermatitis), atopic dermatitis, acne, actinic keratosis, rosacea, a carbuncle, eczema, psoriasis, cellulitis, kertosis, pilaris, melasma, impetigo or a fissure; diminishment or abolishment of scar tissues (for example to treat keloids); breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring

Use of a fully human epidermis according to paragraph 54 or 55 in the manufacture of a medicament for a condition or disease selected from the group consisting of: tissue damage, for example cuts, lacerations, abrasions (such as excoriation), shearing force damage, bites (including animal bites such as dog bites and insect bites); skin regeneration (for example with nerves & organelles); wound healing, for example promoting/enhancing wound healing, including erosions, ulcers (such as diabetic ulcers) wounds from leprosy, wounds from dystrophic epidermolysis, wounds from hidradentitis suppurativa, wounds from mucous membrane pemphigoid, wounds from pemphigoid, wounds from perphigus vulgaris, wounds from pyoderma gangrenosum, wounds from shingles; burn healing including radiation burns, sunburn, chemical burns (such as acid burns and alkali burns), a thermal burn; skin regeneration and repair, for example atrophy, or after excision of tissue, such as where the excision is: cancerous cells skin cells (including melanoma, basal cell carcinoma, squamous cell carcinoma (such as Bowen's disease), extra-mammary Paget's disease), breast cancer, tissue with Darrier's disease, a cyst, a wart (including plantar warts), necrotizing fascilitis (such as methicillin-resistant Staphylococcus aureus, necrotic tissue (such as gangrene), tissue exhibiting Hailey-Hailey disease, tissue exhibiting blisters associated with pemphigus vulgaris; epidermolysis bullosa; enhance skin quality (for example to treat ichthyosis) or appearance; prevention or remediation of skin disorders, for example an abscess, dermatitis (including contact dermatitis), atopic dermatitis, acne, actinic keratosis, rosacea, a carbuncle, eczema, psoriasis, cellulitis, kertosis, pilaris, melasma, impetigo or a fissure; diminishment or abolishment of scar tissues (for example to treat keloids); breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring.

A fully human skin product according to paragraph 54 or 55, for use in testing topical agents.

A kit comprising cell medium according to any one of paragraphs 1 to 18.

A kit according to paragraph 61, further comprising a ROCK inhibitor in a separate container to the medium.

The present inventors established that epidermal growth factor (EGF) and choleratoxin which are present in Green's medium are critical components in Green's medium. Choleratoxin and xenogeneic mouse feeder cells are known to be intimately linked in their mechanism for promoting keratinocyte growth. The present inventors therefore hypothesised that choleratoxin may not act directly on keratinocytes to promote growth but rather stimulate xenogeneic mouse feeder cells to produce keratinocyte growth factor (KGF), thereby indirectly aiding the growth of keratinocytes. Thus, the present inventors elected to replace the need for choleratoxin by adding the end product of choleratoxin stimulation, i.e. KGF, directly into the culture medium.

Accordingly, in one aspect, there is provided a cell culture medium consisting of: DMEM High glucose, Ham's F12, foetal bovine serum, one or more antibiotics and/or antimycotics and keratinocyte growth factor (KGF).

In one embodiment, the cell culture medium consists of: DMEM High glucose:Ham's F12 (3:1), 10% foetal bovine serum, penicillin, streptomycin, 0.625 μg/ml amphotericin B and 20 ng/ml KGF, The present inventors call this medium “Kelch's medium”. Advantageously, the disclosed cell culture medium does not contain choleratoxin. Surprisingly, the inventors have discovered that KGF can be successfully used as a substitute for choleratoxin and when included in a base medium which lacks choleratoxin, is able to provide similar keratinocyte growth kinetics as Green's medium. In addition, the cell culture medium is a minimal medium suitable for supporting the growth of keratinocytes, for example human keratinocytes, which strips out all of the unnecessary components normally present in Green's medium. Further the culture medium of the present disclosure produces similar keratinocyte growth in the presence of mouse embryonic feeder cells (MEFs) as Green's medium with MEFs.

However, to employ unirradiated fibroblast feeder cells then the keratinocytes require the presence a “further” growth acceleration factor. A keratinocyte growth acceleration factor, such as a ROCK inhibitor, is thus generally desirable. This factor can be added as a component of the media or can be used as a component, which is added directly to the culture. Therefore in one embodiment the media is suitable for use with a ROCK inhibitor.

In one embodiment, the cell culture medium consists of: DMEM High glucose:Ham's F12 (3:1), 10% foetal bovine serum, penicillin, streptomycin, 0.625 μg/ml amphotericin B and 20 ng/ml KGF and a keratinocyte growth accelerator factor (such as a ROCK inhibitor in particular one disclosed herein) wherein the factor is other than cholera toxin.

Hence, the presently disclosed cell culture medium can be used in place of Green's medium and without MEFs, thereby eliminating the need for both choleratoxin and MEFs. Furthermore, because the cell culture medium is a minimal medium, it is more convenient and easier to prepare, and also costs less.

In one embodiment, the keratinocyte growth acceleration factor is a ROCK inhibitor, such as a small molecule ROCK inhibitor. Advantageously, the present inventors have established that including a ROCK inhibitor in the cell culture results in enhanced keratinocyte growth rates compared to when ROCK inhibitor is absent.

In one embodiment, the ROCK inhibitor is selected from the group consisting of: SB772077B, Y-27632, Fasudil, Ripasudil, Y39983, Wf-536, SLx-2119, an azabenimidazole-aminofurazan, DE-104, H-1152, ROKα inhibitor, XD-4000, HMN-1152, 4-(1-aminoalkyl)-N-(4-pyridyl)cyclohexane-carboxamide, rhostatin, BA-210, BA-207, BA-215, BA-285, BA-1037, Ki-23095, VAS-012, RKI-1447, GSK429286A, Y-30141, HA-100, H-7, iso H-7, H-89, HA-1004, HA-1077, H-8, H-9, KN-62, GSK269962, and quinazoline.

In one embodiment, the ROCK inhibitor is Y-27632, SB772077B, or a combination of both, in particular SB 772077B. The present inventors have established that both of these ROCK inhibitors are particularly suitable for enhancing keratinocyte growth rates. SB 772077B however has the advantage of having a greater potency and specificity of binding compared to Y27632, which means it can be used at lower concentrations vs Y27632 to achieve the same effect (˜400 nm vs 10 μm).

Also, the addition of ROCK inhibitors to the presently claimed cell culture medium increases the rate of keratinocyte growth to such a significant extent that keratinocytes are able to grow so they are not swamped by fibroblasts when they are grown together in the same culture with unirradiated fibroblasts, in particular unirradiated human fibroblasts.

This is advantageous because in order to grow human keratinocytes from skin samples, traditionally the epidermis must first be separated from the dermis before performing digests to isolate the cells from these 2 layers of skin for in vitro growth in cell culture. The reason for this is that the fibroblasts present in the dermis normally have a tendency to outgrow the keratinocytes when in culture, resulting in a culture of fibroblasts rather than the desired culture of keratinocytes. The ability to grow human keratinocytes together with unirradiated fibroblasts from the same human skin sample greatly reduces the complexity of the sample preparation, by obviating the need to separate the dermis from the epidermis at the beginning of the sample processing. It also allows the preparation of a fully human product.

In addition, because unirradiated human fibroblasts could not previously be cultured together with the human keratinocytes, irradiated xenogeneic feeder cells, for example mouse feeder cells, were usually included in the culture in order to supply the extracellular secretions necessary for the keratinocytes to grow. The need to rely on feeder cells from a different organism is far from ideal when producing keratinocytes for use in skin grafts in the clinic because of the immunogenic potential and the risk of transmitting infectious agents.

The presently disclosed culture medium is able to significantly enhance keratinocyte growth, so unirradiated human fibroblasts can be used as keratinocyte feeder cells instead of irradiated xenogeneic feeder cells, such as MEFs, because the keratinocytes are no longer outgrown by the fibroblasts. Accordingly, the presently disclosed method results in a less immunogenic and less infectious product compared to prior art methods.

In one embodiment, the method further comprises the pre-step of digesting a sample comprising human keratinocytes and human fibroblast feeder cells, such as a skin sample, in the presence of trypsin and optionally collagenase. Advantageously, the present inventors have discovered that digesting skin samples with trypsin only or with trypsin and collagenase results in significantly greater cells yields compared to the typically used prior art dispase collagenase sequential digest method.

Pre-step as employed herein is simply employed to emphasis the step relates to sample preparation and is not intended to put restrictions on the order that steps are performed herein. Thus, the steps of the method can be performed in any reasonable order, which provides the skin product.

In one embodiment, both keratinocytes and fibroblast cells are isolated from the same skin sample in a single digestion reaction. Advantageously, the disclosed method allows keratinocyte cells to be isolated from the epidermis and fibroblast cells to be isolated from the dermis using a single digestion reaction. This eliminates the requirement for the epidermis and dermis to be digested in two separate reactions, thereby saving time and simplifying the process.

In one embodiment, the sample is digested in the presence of both trypsin and collagenase. The advantage of digesting in the presence of collagenase is that the collagenase helps to digest the dermis and basement membrane in order to release more cells from the skin tissue. This may result in even higher cell yields compared to when trypsin alone is used. Furthermore, the present inventors have found that keratinocytes and/or fibroblasts isolated by digesting skin in both trypsin and collagenase may proliferate faster when grown in culture compared to keratinocytes and/or fibroblasts isolated from dispase digested epidermis and collagenase digested dermis.

In one embodiment, the collagenase is type I collagenase. Advantageously, type I collagenase has collagenase, caseinase, clostripain and tryptic activities, making it well suited for the digestion of a skin sample which comprise a range of different cell types.

In one embodiment, the skin sample is obtained from a human (in particular a patient), from any suitable location, for example a shoulder, arm, thigh, calf, breast, abdomen, foreskin and/or buttocks.

In one embodiment, the human keratinocytes are not cultured in the presence of xenogeneic feeder cells. This has the advantages discussed above. In one embodiment, the human fibroblast cells are not irradiated. The advantage of this is that the fibroblast cells are still active and are ultimately able to able to grow and differentiate into a dermis. Another advantage is that the process of growing keratinocytes is greatly simplified since there is no need to irradiate cells, or to ensure through quality control systems that the irradiation process has not allowed any cells to escape irradiation and proliferate.

In one embodiment, the human fibroblasts are autologous to a human patient. In one embodiment, the human keratinocytes are autologous to a human patient. Advantageously, this significantly reduces the immunogenic risk for a patient when the fully human epidermis is generated from the patient's own skin cells. Furthermore, the skin graft product prepared by the method of the present disclosure can be produced in as little as 3 weeks, which makes it viable to generate grafts specific for an individual patient. Autologous and allogeneic (also referred to as heterologous) is relevant in the context of applying the skin graft to a particular patient and perhaps in the context of obtaining the original sample. It is not a parameter that the influences the individual steps of the method per se.

In one embodiment, the keratinocytes are cultured for a period without contacting a gas permeable membrane (interface). In one embodiment, the keratinocytes are cultured, for a period, in contact with a gas permeable membrane (interface). The latter helps the cells differentiate. In one embodiment, the period of culture where the keratinocytes are in contact with the gas permeable membrane follows the period of culturing without contacting a gas permeable layer.

In one embodiment, the distance of the relevant cells to the gas permeable layer (for example wherein the cells are in contact with the gas permeable layer) is 2 cm or less, such as 1 cm or less, in particular 0.5, 0.4, 0.3, 0.2, 0.1 or 0.05 cm.

In one embodiment, the substrate is coated with an extracellular matrix protein or peptide thereof, for example a synthetic peptide (such as collagen, laminin and other extracellular matrix proteins). Advantageously, the presence of the coating produces a second cellular signal (the first signal being growing the skin tissue at an air-liquid or gas permeable interface), which enhances the proper stratification of the skin tissue.

As discussed above in one embodiment the he substrate is located in a culture device comprising a gas permeable membrane, for example the substrate is moveable to a position where cells deposited thereon are in contact with a gas permeable layer, or a where said cells are not in contact with a gas permeable layer. A suitable device is disclosed in WO2016/209089, incorporated herein by reference.

In one embodiment, the culture device comprises: a container comprising a first endwall (bottom), and at least one sidewall, a detachable second endwall (top) adapted to engage with the container to define a chamber, and a scaffold adapted to receive a substrate for cells to reside upon, wherein at least a part of at least one of the first endwall (bottom), the at least one sidewall, or the second endwall (top) comprises a gas permeable material or is adapted to engage with a gas permeable material and is perforated to allow gaseous exchange; and wherein the device is configurable between (a) a first mode in which the substrate is not disposed in gaseous communication with a gas permeable material, and (b) a second mode in which the substrate is moved to be disposed in gaseous communication with a gas permeable material.

Advantageously, the ability to move the substrate between the two modes allows the cells to be submerged in media during the initial phase of growth and then easily put in contact with the atmosphere (such as contacting a gas permeable membrane) for proper differentiation into full thickness skin.

In one embodiment, the scaffold engages with the at least one sidewall to (a) allow substantially linear movement of the scaffold at least partway between the first endwall (bottom) of the chamber and the second endwall (top), and restrict rotation or inversion of the scaffold about an axis perpendicular to the at least one sidewall, or (b) allow rotational movement of the scaffold about an axis perpendicular to the at least one sidewall.

In one embodiment, the scaffold comprises a frame defining an interior perimeter and an exterior perimeter, said frame comprising a substantially planar upper surface, a substrate for cells to reside upon held in a substantially planar arrangement across the interior perimeter of the frame, wherein the scaffold is configured to bring substantially all of the substrate or the cells or tissues present on the substrate into contact with a gas permeable interface when the scaffold is placed in a culture apparatus comprising at least one gas permeable interface.

In one aspect, there is provided a human full thickness skin, for example comprising both an epidermis and a dermis, cultured by the method as described herein.

In one aspect, there is provided a fully human epidermis cultured by the method as described herein.

In one aspect, there is provided a human full thickness skin, a fully human epidermis or a fully human dermis cultured by the method as described herein for use in treatment, in particular there is provided a fully human skin product comprising an epidermis.

In one embodiment, the treatment is for a condition or disease selected from the group consisting of: tissue damage; skin regeneration with nerves & organelles; wound healing, for example promoting/enhancing wound healing, including ulcers such as diabetic ulcers; burn healing; skin regeneration and repair; epidermolysis bullosa; enhance skin quality or appearance; prevention or remediation of skin disorders; diminishment or abolishment of scar tissues; breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring.

In one aspect, there is provided a use of a human full thickness skin, a fully human epidermis or a fully human dermis cultured by the method as described herein in the manufacture of a medicament for a condition or disease disclosed herein for example above.

In one aspect, there is provided a method of treatment comprising suturing a human full thickness skin, a fully human epidermis or a fully human dermis cultured by the method as described herein to a patient in need thereof.

In one embodiment, the treatment is for a condition or disease, for example disclosed elsewhere herein.

### BRIEF DESCRIPTION OF THE DRAWINGS

FIG. 1 shows that several components in Green's medium are not required for keratinocyte growth in the presence of a ROCK inhibitor. Human primary keratinocytes were recovered from cryostorage and grown in Green's media, Kelch's media (“base media”+KGF in the figure), or DF10, all supplemented with the ROCK inhibitor Y27632, until 80% confluence was reached. To identify dispensable components of Green's media, one component at a time was left out of the Green's mixture. KGF was added to identify its effect on keratinocyte growth in each media. Keratinocytes were either grown in the presence of mouse embryonic feeder cells (MEFs) (right) or without (left). For evaluation of keratinocyte growth cells were fixed, stained with rhodamine B, and confluency was measured using the Operetta plate reader. Results are shown as triplicates for one representative donor (+SD), the level of growth in Green's media+Y27632 is indicated by a dashed line. N=2. These results show that even in the absence of MEFs, when supplemented with ROCK inhibitor, Kelch's medium is as effective at inducing keratinocyte proliferation as Green's medium. It also shows that both cholera toxin and EGF are important components of Green's medium in generating keratinocyte proliferation.

FIG. 2 shows the results of a comparison between Keratinocyte growth in Kelch's medium vs. Green's medium. Human primary keratinocytes from frozen epidermal preparations were grown to 80% confluence in different media supplemented with the ROCK inhibitor SB772077B. Cells were grown in conventional Green's media with or without MEFs (right), or using Base medium supplemented with KGF, MEFs, or both (left). Kelch's medium is represented by Base medium plus KGF in the figure. For evaluation of keratinocyte growth cells were fixed, stained with rhodamine B, and confluency was measured using the Operetta plate reader. Results are showing the average of 3 donors (+SD) and statistical analysis was performed using two-tailed student's t test. N=3. These results show that Kelch's medium generates keratinocyte proliferation at least as well as Green's medium, either in the presence or absence of MEF feeder cells. It also shows that KGF is a critical component in Kelch's medium, because keratinocyte proliferation in Base medium plus ROCK inhibitor is significantly lower than in Base medium plus ROCK inhibitor plus KGF. Inclusion of KGF in Kelch's medium in the absence of feeder cells enables keratinocyte proliferation that is statistically equivalent to proliferation in Green's medium in the presence of feeder cells, confirming that Kelch's medium enables omission of xenogeneic feeder cells.

FIG. 3 shows that ROCK inhibitors Y27632 and SB772077B have comparable effects on keratinocyte growth. Frozen human keratinocytes were grown in Base medium, Base medium plus KGF (“Kelch's medium” in the figure legend), or Green's medium, supplemented with either Y27632 or SB772077B. For evaluation of keratinocyte growth cells were fixed, stained with rhodamine B, and confluency was measured using the Operetta plate reader. Results are shown for 3 donors in triplicate (+SD). N=3. These results show that when supplemented with a ROCK inhibitor, Kelch's medium (Base medium plus KGF)) drives superior keratinocyte proliferation to either Green's medium or Base medium plus ROCK inhibitor without KGF, demonstrating that KGF is a crucial component of Kelch's medium.

FIG. 4 shows that Kelch's medium plus ROCK inhibitor SB772077B) provides equivalent keratinocyte growth to Green's medium plus a ROCK inhibitor. T rypsin and collagenase digested skin cells were grown in Kelch's medium v Greens medium, both containing ROCK inhibitor SB772077B. Triplicate samples were grown for up to 30 days, and were passaged 1:9 when they reached 80-90% confluency. N=1.

FIG. 5 shows the effect of serum-free Optipeak medium (InVitria) on Keratinocyte growth. Human primary keratinocytes from cryopreserved epidermal preparations were grown in 4 different formulations of serum-free Optipeak media or Green's medium with mouse embryonic feeder cells (MEFs) for 8 days. All media were tested with and without ROCK inhibitor Y27632. For evaluation of keratinocyte cell growth cells were fixed, stained with rhodamine B, and confluency was measured using the Operetta plate reader. Results are shown as triplicates of one representative donor (+SD). N=2. These results show that while ROCK inhibitor enhanced growth of keratinocytes in serum-free Optipeak medium, the resulting proliferation was still inferior to that resulting from culture in Green's medium in the presence of MEFs. These results also show that ROCK inhibitor can enhance growth of keratinocytes in Green's medium in the presence of MEFs.

FIG. 6 shows a comparison of keratinocyte growth rates between Greens medium and commercially available keratinocyte growth media. Epidermal cells were grown in Greens medium with MEFs, CnT Prime, Epilife medium supplemented with S7 or EDGS. All media were tested with and without ROCK inhibitor Y27632. 550,000 epidermal cells were grown in each medium until one sample reached 80% confluence, at this point all samples were fixed and stained for rhodamine B. Sample confluency was then assigned a score between 0 and 4, 0 having no cells, and 4 having maximum confluency. N=2. These results indicate that ROCK inhibitor may also enhance growth of keratinocytes in other keratinocyte growth media, but that none of these media can produce equivalent growth of keratinocytes to Green's medium plus MEF, unlike Kelch's medium.

### EXAMPLES

CnT-Prime is a fully defined, animal-component-free culture medium available from CellnTec. It is suitable for isolation and expansion of epithelial cells from skin, cornea, gingiva, mammary and bladder tissue. It was developed using human tissue, but may also be used with other species (e.g. mouse).

Supplement S7 is available from ThermoFisher. It is a sterile, concentrated (100×), ionically balanced solution intended for use as one component in a complete culture environment for human epidermal keratinocytes. Supplement S7 is chemically defined and animal origin-free. Each 5 ml bottle of Supplement S7 is the correct amount of supplement for a 500 ml bottle of EpiLife® basal medium.

EDGS is also available from ThermoFisher. It is a defined, sterile, concentrated (100×) solution intended for use with EpiLife® medium to culture human epidermal keratinocytes. EDGS is not intended for use with Medium 154. EDGS contains all of the growth factors and hormones necessary for the growth of human epidermal keratinocytes. Each bottle contains the correct amount of supplement for a 500 ml bottle of EpiLife® medium. EDGS contains: •purified bovine serum albumin; •purified bovine transferrin; •hydrocortisone; •recombinant human insulin-like growth factor type-1 (rhIGF-1); •prostaglandin E2 (PGE2); •recombinant human epidermal growth factor (rhEGF). The bovine products in EDGS have been isolated from animals of North American origin. The isolation process for these components includes steps that inactivate viruses. All components of EDGS are greater than 95% pure

### Example 1—Methods

Materials

Greens medium—DMEM High glucose:Hams F12 3:1, 10% foetal bovine serum, penicillin, streptomycin, 10 ng/ml EGF, 0.1 nM choleratoxin, 0.4 μg/ml hydrocortisone, 180 μM adenine, 5 ug/ml insulin, 5 μg/ml apotransferrin, 2 nM 3,3,5,-tri-idothyronine, 2 mM glutamine, 0.625 μg/ml Amphotercin B, 10 μM Y27632

Kelch's medium—DMEM High glucose:Hams F12 3:1, 10% foetal bovine serum, penicillin, streptomycin, 0.625 μg/ml amphotericin, 20 ng/ml KGF

Base medium—DMEM High glucose:Hams F12 3:1, 10% foetal bovine serum, penicillin, streptomycin, 0.625 μg/ml amphotericin

Y27632 used at 10 μm final concentration. SB772077B used at 400 nM final concentration.

CnT Prime—Cellntech catalogue number CNT-PR; Epilife medium—Thermofisher catalogue number MEPI500CA; S7 supplement—Thermofisher catalogue number 50175; EDGS—Thermofisher catalogue number 50125; Coating matrix—Thermofisher catalogue number R011K; Y27632—Selleckchem catalogue number S1049; SB772077B—Tocris catalogue #4118

Tryple—Life Technologies catalogue #12604021

Greens, Cellntech, S7, EDGS Medium Comparison

Flasks were pre-coated with a coating matrix for S7 and EDGS supplemented Epilife medium samples. 1.7 ml of dilution buffer was added to each T25 flask. Then 17 μl of coating matrix was added. The mixture was incubated at room temperature for 30 minutes. Next, the dilution buffer and coating matrix mix were removed from the flasks before adding cells and media.

550,000 epidermal cells and 5 ml of each required medium was added, with or without 10 μl Y27632, to a T25 flask. The flask was incubated at 37° C. with 5% CO₂ until one sample reached 80% confluency.

Next, the medium was removed from the flasks and the cells fixed with 4% formaldehyde for 30 minutes. 1% rhodamine B solution was added and the cells incubated at room temperature for 10 minutes. The rhodamine B solution was removed and the cells thoroughly rinsed with water. Finally, all liquid from the flasks was removed using a pipette. The cells were imaged and confluency was calculated.

Trypsin and Collagenase Digest Method

All subcutaneous fat and hypodermis was trimmed away from skin samples. Next, the skin was cut into 1 cm² pieces and weighed. Finally, the underside of the dermis was heavily scored using a scapel.

The pieces of skin were placed in the wells of a 6 well plate. Next, 5 mg/ml collagenase (type I) was prepared with MilliQ water and the enzyme stock was filtered through a 0.2 um syringe filter in hood. 0.25% trypsin was then diluted to 0.1% in DO and 600 μl of collagenase was added, making a total volume of 6 ml. The plate was placed in an incubator at 37° C. with 5% CO₂ overnight.

The next day, the skin and enzyme containing medium was transferred to 6 ml of DF10 in a 10 cm dish and the skin teased apart with a scalpel. The skin was next broken up further by passing through a pipette repeatedly. Next, the cell mix was passed through a 100 μm strainer.

Next, the mix was centrifuged at 1800 rpm for 10 min and as much supernatant as possible was removed without disturbing cell pellet. Finally, the cell pellets were resuspended in DF10 to assess cell number and viability.

Keratinocyte Expansion

4.5 ml of keratinocyte growth medium with 400 nM SB 772077B was added to a T25 flask.

500 μl of single cell suspension from the trypsin/collagenase digest process was added into the T25 flask. The cells were then incubated overnight at 37° C./5% CO₂.

All medium was removed and replaced with 5 ml of keratinocyte growth medium with 400 nM SB772077B.

Cell growth was monitored until the flask reaches 80-90% confluence, then the cells were passaged for continued expansion.

Passage 1 Keratinocyte Passage

All medium was removed from the T25 flask.

The cells were washed once with 1 ml of TrypLE™. 2 ml of Tryple was added and the mixture incubated at 37° C./5% CO₂ until the majority of the cells detached when the flask was struck firmly.

4 ml of keratinocyte growth medium with 400 nM SB772077B was added to the T25 flask. Next, the base of the flask was rinsed 3 times using a serological pipette. This detached any remaining cells and breaked up clumps of cells.

10 ml of keratinocyte growth medium with 400 nM SB772077B was added to a T75 flask. One third of the detached cell mix was added to the T75 flask.

Next, the cells were incubate at 37° C./5% CO₂. Cell growth was monitored until flask reached 80-90% confluence then the cells were passaged for continued expansion.

P2 to Px Keratinocyte Passage

All medium was removed from the T75 flask. Next, the cells were washed once with 2 ml of TrypLE™. 4 ml of TrypLE™ was added and the cells incubated at 37° C./5% CO₂ until the majority of cells detached when the flask was struck firmly.

Next, 8 ml of keratinocyte growth medium with SB772077B was added to the T75 flask. The base of the flask was then rinsed 3 times using a serological pipette. This detached any remaining cells and broke up any clumps of cells.

11 ml of keratinocyte growth medium with 400 nM SB772077B was added to a T75 flask. One tenth of the detached cell mix was then transferred to the T75 flask.

The cells were incubated at 37° C./5% CO₂. Cell growth was monitored until flask reached 80-90% confluence before the cells were passaged for continued expansion.

### Example 2—Determining which Components in Green's Medium were not Required for Keratinocyte Growth

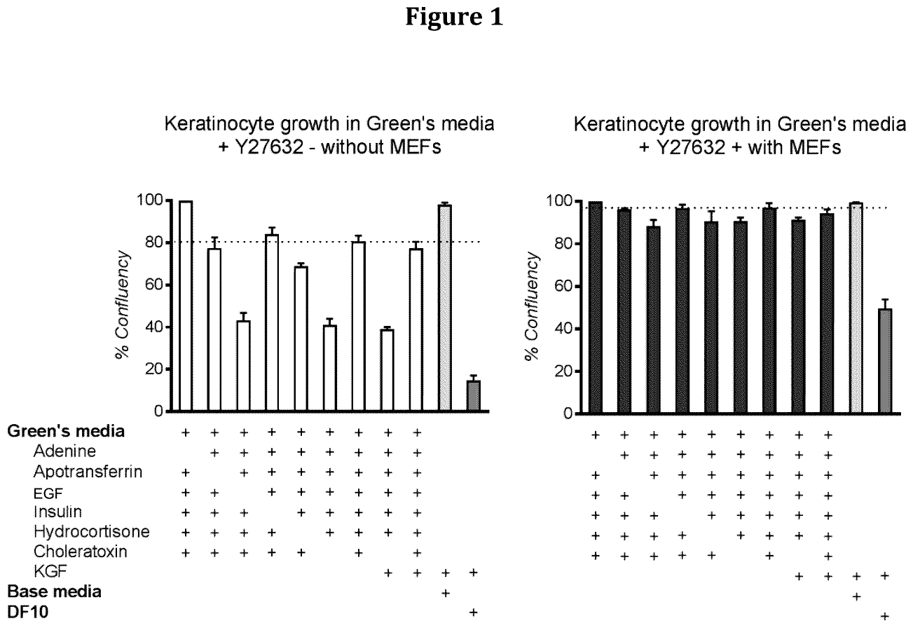

In FIG. 1 the active components of Green's media are evaluated by leaving them out of the media mixture one at a time. Importantly, cells are grown in the presence of ROCK inhibitor (Y27632), which may stimulate cell growth to a point where the effect of some Green's media components becomes obsolete. The graph on the right shows that if keratinocytes are co-cultured with mouse embryonic feeder cells (MEFs), they grow rapidly in either mixture. The lack of any individual ingredient does not seem to have a significant effect. However, if MEFs are not present (left graph), the lack of certain ingredients seems to negatively affect keratinocyte growth, namely epidermal growth factor (EGF) & choleratoxin. We later found that EGF is not enhancing keratinocyte growth (data not shown).

Taken together, choleratoxin appears to be one of the most important single ingredients of Green's media. Interestingly, keratinocyte growth factor (KGF) stimulates growth when added to base media (DMEM:F12 3:1, 10% foetal bovine serum, penicillin, streptomycin, amphotericin)+Y27632, but fails to enhance keratinocyte growth in Green's media+Y27632, and cannot rescue the growth in the media lacking choleratoxin. Overall, keratinocyte growth in Base media+KGF+Y27632 (Kelch's medium) is similar if not better than growth in Green's media Y27632.

Removal of adenine alone from Green's medium+Y27632 appears to enhance growth of keratinocytes.

Keratinocytes also appear to grow considerably better in Green's or Kelch's media compared to DF10+KGF.

### Example 3—Comparison of Keratinocyte Growth in Kelch's Medium Vs Green's Medium

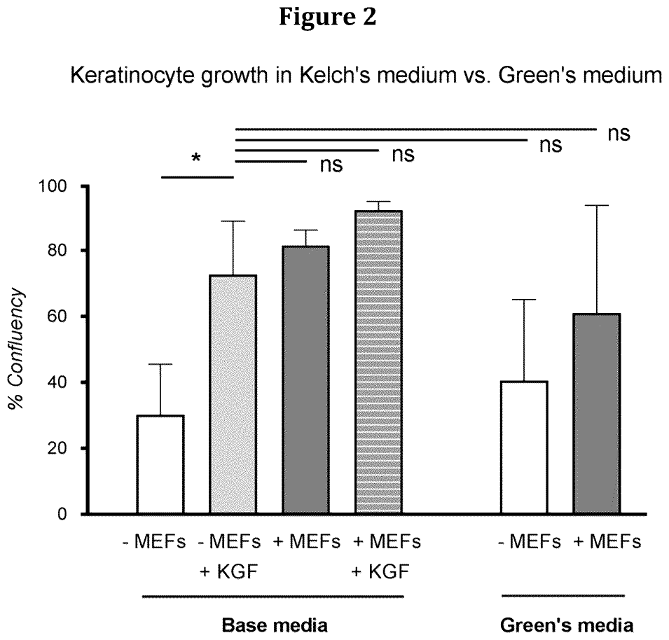

FIG. 2 compares keratinocyte growth in Kelch's medium and Green's medium, with both media supplemented with ROCK inhibitor. Keratinocytes appear to grow best in the presence of MEFs, rather than media alone, which is true for both Kelch's and Green's media. In the presence of ROCK inhibitor, adding KGF to Base medium (Base medium+KGF=Kelch's medium) has a similar effect as adding MEFs to Base medium, suggesting that KGF is a potent substitute for MEFs in keratinocyte culture. Keratinocyte growth in Kelch's medium is equivalent to Green's media (+/−MEFs). Keratinocyte growth in Kelch's medium with ROCK inhibitor is significantly better than Base medium with ROCK inhibitor lacking KGF, but not significantly different from Kelch's media+MEFs+ROCK inhibitor or Green's media+ROCK inhibitor (+/−MEFs).

Thus, Kelch's medium appears to produce similar keratinocyte growth rates compared to Green's medium.

### Example 4—Comparison of Keratinocyte Growth Using Y27632 vs SB772077B

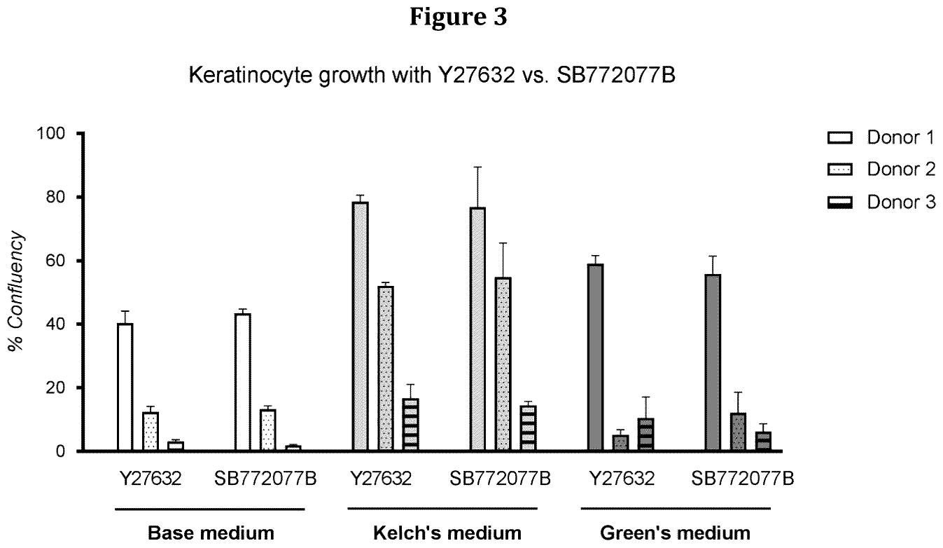

FIG. 3 shows the similarities of the effects of ROCK inhibitors Y27632 and SB772077B on keratinocyte growth. Therefore, SB772077B can be used as substitute for Y27632 for keratinocyte growth.

### Example 5—Comparison of Keratinocyte Growth Obtained from Trypsin Collagenase Digested Skin in Kelch's Medium Vs Green's Medium

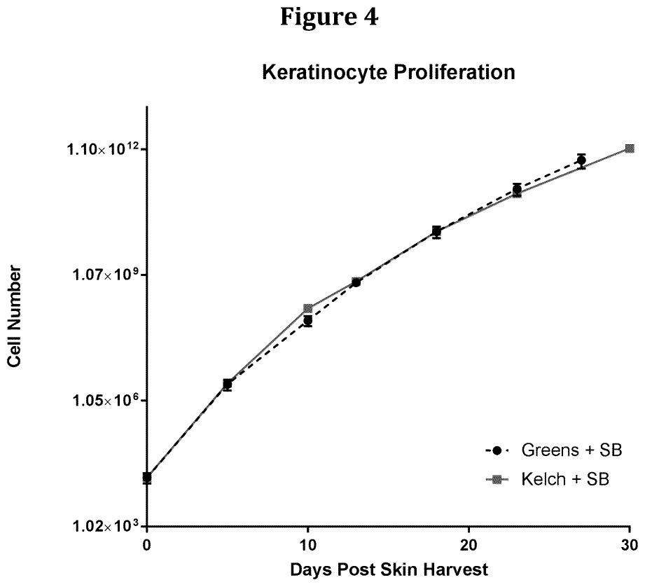

Skin samples digested using trypsin and collagenase were grown in Green's medium supplemented with SB772077B or Kelch's medium supplemented with SB772077B. FIG. 4 shows a graph of keratinocyte proliferation rates. Over a period of 30 days keratinocyte growth in Kelch's medium was equivalent to keratinocyte growth in Green's medium. Thus, Kelch's medium appears to be a good replacement for Green's medium since it can provide equivalent keratinocyte growth kinetics and does not require choleratoxin.

### Example 6—Experiment to Determine Potential Use of Serum-Free Opti-Peak™ Media

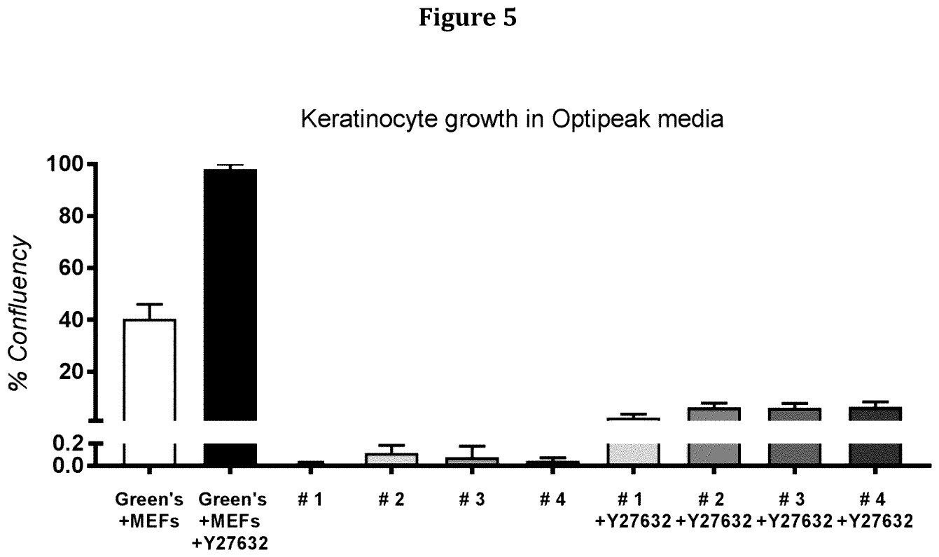

FIG. 5 examines the potential use of serum-free Optipeak media for keratinocyte growth. We tested 4 formulations of Opti-Peak™ media provided by InVitria. In either of these media alone, cells did not exhibit any noticeable growth. If ROCK inhibitor Y27632 was added to the culture, a few colonies started to develop. However, none of the 4 formulations seem to be a good match for Green's medium, which by itself and especially in the presence of Y27632 is significantly more potent for keratinocyte growth.

### Example 7—Comparison of Keratinocyte Cell Growth Between Green's Medium and Commercial Keratinocyte Growth Media

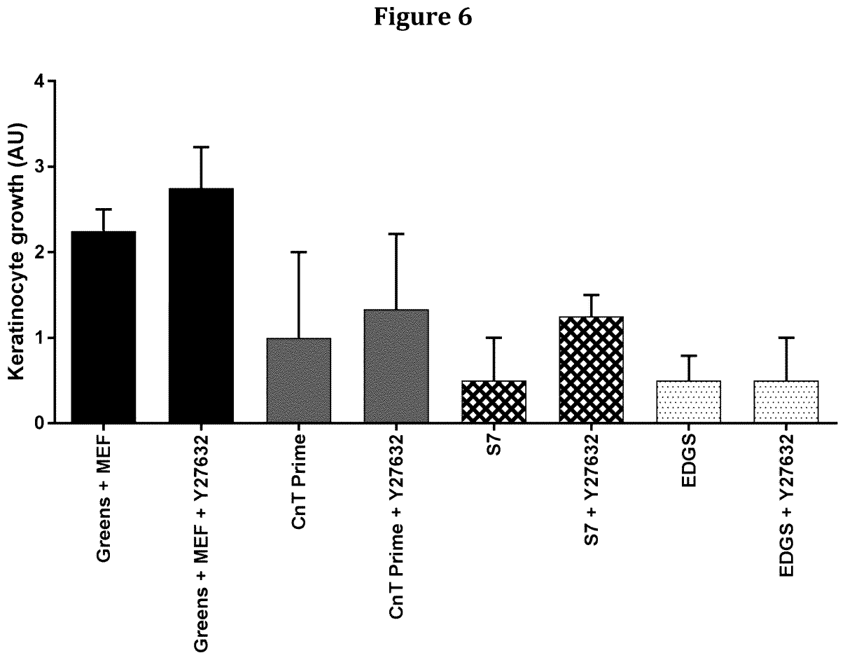

FIG. 6 shows a comparison of keratinocyte cell growth between Green's medium and commercial keratinocyte growth media. S7 and EDGS are used to supplement EpiLife® medium, from the Gibco range. CnT Prime was obtained from Cellntech. All commercial media were designed to be serum and feeder free. Green's medium uses both serum and mouse embryonic feeder cells. All media were used with and without ROCK inhibitor Y27632, known to have a positive effect on keratinocyte growth. Green's medium supplemented with ROCK inhibitor and mouse embryonic feeder cells provided the fastest keratinocyte growth. ROCK inhibitor enhanced keratinocyte growth in Green's medium, S7 medium and CnT Prime medium.

### DETAILED DESCRIPTION OF THE PREFERRED EMBODIMENT

“DMEM” or “Dulbecco's Modified Eagle Medium” is a modification of Basal Medium Eagle which contains a four-fold higher concentration of amino acids and vitamins, together with additional components. The specific ingredients are listed in the background section herein. DMEM normally contains about 1000 mg/L of glucose. “DMEM high glucose” refers to a version of DMEM which contains 4500 mg/L of glucose instead of the usual 1000 mg/L.

“Ham's F12” is a medium designed for low density, serum-free growth of Chinse Hamster Ovary (CHO) cells. Ham's F12 is based on Ham's F10 medium but with increased concentrations of choline, inositol, putrescine and other amino acids. The specific ingredients are listed in the background section herein.

The ratio of DMEM:Ham's F12 in the cell culture medium may be 1:1, 2:1, 3:1, 4:1, 5:1. In one embodiment, the ratio is 3:1.

Green's media as employed herein is DMEM:Hams F12 (Life Technologies 31765-035) at a ratio of 3:1. Fetal calf serum (FCS) is generally used as the serum, although NCS and serum substitute products are also suitable, while Hepes buffer, for example, is used as the buffer. The pH value of the solution of cell culture medium, buffer and serum is usually in the range from 6.0 to 8.0, for example, from 6.5 to 7.5 and, more particularly, 7.0.

“Foetal bovine serum” or FBS is the most widely used animal serum supplement for the culture of eukaryotic cells. This is due to the very low level of antibodies and presence of growth factors which makes FBS suitable for many different cell culture applications. The presently claimed culture medium contains foetal bovine serum but the skilled person would be aware that other types of serum can also be used, for example bovine serum albumin (BSA), human platelet lysates and iron-supplemented bovine calf serum (ICS). 10% serum is typically used but other concentrations may also be used depending on the type of serum, for example 0.1% to 20%, such as 0.5%, 1%, 1.5%, 2%, 3%, 4% 5%, 6%, 7%, 8%, 9%, 10%, 11%, 12%, 13%, 14%, 1, 1⁶%, 1⁷%, 18%, 19% and 20%.

“Penicillin” as used herein refers to a group of β-lactam antibiotics, including penicillin G, penicillin V, procaine penicillin and benzathine penicillin. Penicillin acts by inhibiting the formation of peptidoglycan cross-links in the bacterial cell wall. This weakens the cell walls of dividing bacterial, eventually causing the cell walls to burst and the bacteria to die because of osmostic pressure. Gram-positive bacteria have thick cell walls containing high levels of peptidoglycan, whereas gram-negative bacteria are characterised by thinner cell walls with low levels of peptidoglycan. Thus, penicillin is most effective against gram-positive bacteria.

“Streptomycin” is an antibiotic that was originally purified from Streptomyces griseus. It acts by binding to the 30S subunit of the bacterial ribosome, leading to inhibition of protein synthesis and death in susceptible bacteria. Streptomycin is able to cross the outer cell wall of negative organisms by passive diffusion through aqueous channels. Conversely, the thicker cell walls of gram-positive bacteria inhibits transport of streptomycin. Accordingly, streptomycin works better on gram-negative bacteria.

In one embodiment cell culture medium contains both penicillin and streptomycin, thereby helping to protect the cells grown in the culture from both gram-positive and gram-negative bacteria.

“Gentamicin” is an antibiotic comprising a complex of three different closely rated aminoglycoside sulfates, Gentamicins C1, C2 and C1a, obtained from Micromonospora purpurea and related species. Gentamicin is a broad spectrum antibiotic typically used for serious infections of the following microorganisms: P. aeruginosa, Proteus species (indole-positive and indole-negative), E. coli, Klebsiella-Enterobactor-Serratia species, Citrobacter species and Staphylococcus species (coagulase-positive and coagulase-negative).

In one embodiment, the cell culture medium contains gentamicin.

In one embodiment the medium does not contain an antibiotic or antibiotics, which are associated with allergic reactions, such as penicillin and/or streptomycin.

“Amphotericin B” is an anti-fungal medication used for serious fungal infections and leishmaniasis. It functions by binding with ergosterol, a component of fungal cell membranes, forming pores that case rapid leakage of monovalent ions (eg. K⁺, Na⁺, H⁺ and Cl⁻), which leads to fungal cell death.

In one embodiment, the cell culture medium contains an antibiotic which targets gram-positive bacteria, such as penicillin, an antibiotic which targets gram-negative bacteria, such as streptomycin, and an anti-fungal medication.

“Keratinocyte growth factor” or KGF, is a growth factor present in the epithelialization-phase of wound healing. KGF is encoded in humans by the FGF7 gene. KGF is a small signaling molecule that binds to fibroblast growth factor receptor 2b (FGFR2b). There are 23 known FGFs, and 4 FGF receptors. KGF is known to be a potent epithelial cell-specific growth factor, whose t mitogenic activity is predominantly exhibited in keratinocytes but not in fibroblasts or endothelial cells.

“ROCK inhibitor” as used herein refers to any compound or protein which has a function in reducing or blocking the activity of Rho-associated protein kinase (ROCK), for example a small molecule ROCK inhibitor or an antibody.

Examples of ROCK inhibitors include but are not limited to: SB 772077B, Y-27632, Fasudil, Ripasudil, Y39983, Wf-536, SLx-2119, an azabenimidazole-aminofurazan, DE-104, H-1152, ROKα inhibitor, XD-4000, HMN-1152, 4-(1-aminoalkyl)-N-(4-pyridyl)cyclohexane-carboxamide, rhostatin, BA-210, BA-207, BA-215, BA-285, BA-1037, Ki-23095, VAS-012, RKI-1447, GSK429286A, Y-30141, HA-100, H-7, iso H-7, H-89, HA-1004, HA-1077, H-8, H-9, KN-62, GSK269962, and quinazoline.

| | |
|---|---|
| In one embodiment the ROCK inhibitor is SB772077B, also known as (3S)-1-[[2-(4-Amino-1,2,5-oxadiazol- 3-yl)-1-ethyl-1H-imidazo[4,5-c]pyridin- 7-yl]carbonyl]-3-pyrrolidinamine dihydrochloride: | 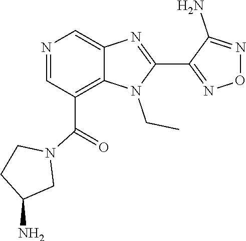 [SMILES: CCn1c(-c2nonc2N)nc2cncc(C(=O)N3CC[C@H](N)C3)c21] |
| In another embodiment the ROCK inhibitor is a compound of the general formula (I): wherein: R¹ and R² can connect through an aromatic group or R¹ is hydrogen or an alkyl, and R¹ and R² are not the same unless they are part of a cyclic aromatic, and R³ and R⁴ are attached to any atom in the isoquinoline and are independently a hydrogen or an alkyl. | 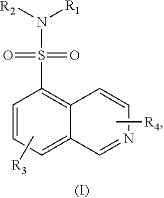 |

In one embodiment, the ROCK inhibitor is
H-1152:

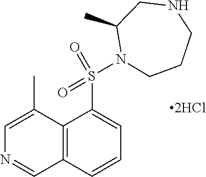 [SMILES: Cc1cncc2cccc(S(=O)(=O)N3CCCNC[C@@H]3C)c12]

In one embodiment, the ROCK
inhibitor is HA-100:

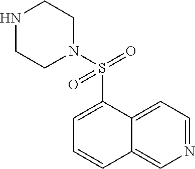 [SMILES: O=S(=O)(c1cccc2cnccc12)N1CCNCC1]

In one embodiment, the ROCK inhibitor is
H-7:

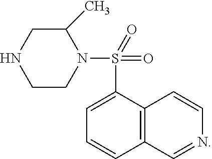 [SMILES: CC1CNCCN1S(=O)(=O)c1cccc2cnccc12]

In one embodiment, the ROCK
inhibitor is H-8:

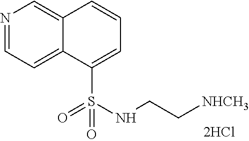 [SMILES: CNCCNS(=O)(=O)c1cccc2cnccc12]

In one embodiment the ROCK inhibitor is
Y-27632:

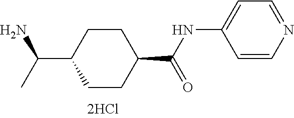 [SMILES: C[C@@H](N)[C@H]1CC[C@H](C(=O)Nc2ccncc2)CC1]

In one embodiment, the ROCK
inhibitor is HA-1004:

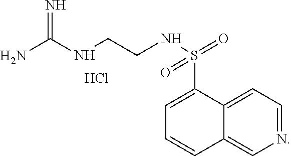 [SMILES: N=C(N)NCCNS(=O)(=O)c1cccc2cnccc12 ; Cl]

In one embodiment, the
ROCK inhibitor is
H-89:

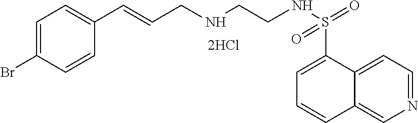 [SMILES: O=S(=O)(NCCNC/C=C/c1ccc(Br)cc1)c1cccc2cnccc12]

In one embodiment, the ROCK
inhibitor is Fasudil:

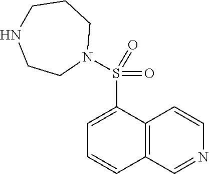 [SMILES: O=S(=O)(c1cccc2cnccc12)N1CCCNCC1]

In one embodiment, the ROCK inhibitor is
HA-1077:

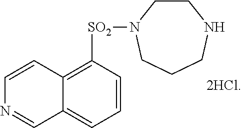

In one embodiment, the ROCK
inhibitor is H-9:

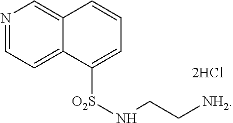 [SMILES: NCCNS(=O)(=O)c1cccc2cnccc12]

In one embodiment, the ROCK inhibitor is
KN-62:

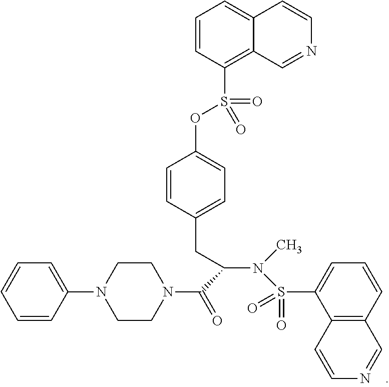 [SMILES: CN([C@@H](Cc1ccc(OS(=O)(=O)c2cccc3ccncc23)cc1)C(=O)N1CCN(c2ccccc2)CC1)S(=O)(=O)c1cccc2cnccc12]

In one embodiment, the ROCK
inhibitor is H-1152:

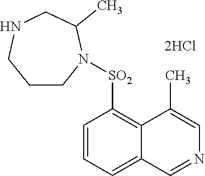 [SMILES: Cc1cncc2cccc(S(=O)(=O)N3CCCNCC3C)c12]

In one embodiment, the ROCK inhibitor is
GSK269962:

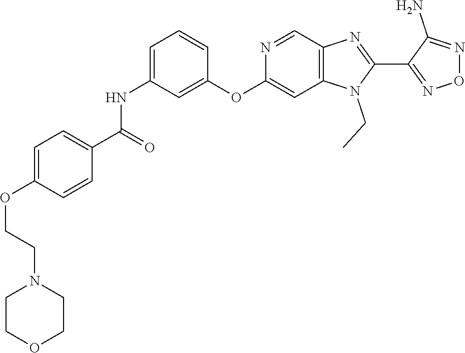 [SMILES: CCn1c(-c2nonc2N)nc2cnc(Oc3cccc(NC(=O)c4ccc(OCCN5CCOCC5)cc4)c3)cc21]

In one embodiment, the ROCK inhibitor is
GSK429286:

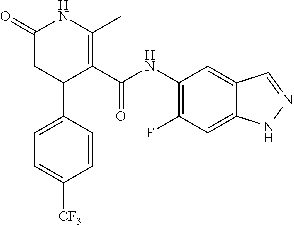 [SMILES: CC1=C(C(=O)Nc2cc3cn[nH]c3cc2F)C(c2ccc(C(F)(F)F)cc2)CC(=O)N1]

In one embodiment, the ROCK inhibitor is Ripasudil:

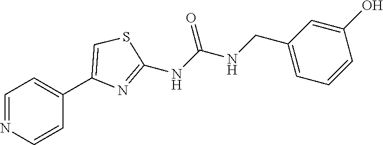 [SMILES: O=C(NCc1cccc(O)c1)Nc1nc(-c2ccncc2)cs1]

The skilled person is aware of other ROCK inhibitors.

The terms “epithelia” and “epithelium” refer to the cellular covering of internal and external body surfaces (cutaneous, mucous and serous), including the glands and other structures derived therefrom, e.g., corneal, esophageal, laryngeal, epidermal, hair follicle and urethral epithelial cells.

In one embodiment, the cells employed are skin cells, such as human skin cells, for example cells which form an epidermis and dermis, such as fibroblasts and keratinocytes.

Other exemplary epithelial tissues include: olfactory epithelium, which is the pseudostratified epithelium lining the olfactory region of the nasal cavity, and containing the receptors for the sense of smell; glandular epithelium, which refers to epithelium composed of secreting cells; squamous epithelium, which refers to epithelium composed of flattened plate-like cells.

The term “tissue” is used to refer to an aggregation of similarly specialized cells united in the performance of a particular function. Tissue is intended to encompass all types of biological tissue including both hard and soft tissue. A “tissue” is a collection or aggregation of particular cells embedded within its natural matrix, wherein the natural matrix is produced by the particular living cells. The term may also refer to ex vivo aggregations of similarly specialized cells which are expanded in vitro such as in artificial organs.

The term “skin tissue,” or “skin” as used herein, refers to any tissue, including epidermis, dermis and may also include basement membrane tissue, for example full thickness skin.

The method according to the present disclosure is capable of generating full thickness human skin, which is made up of two tissue-specific layers, namely a dermis equivalent and an epidermis equivalent. The skin tissue substantially corresponds to native skin both histologically and functionally.

As used herein “epidermis” refers to the outer of the two layers which make up the skin, the inner layer being the “dermis”.

As used herein “fully human” refers to a tissue which does not comprise and was not prepared any non-human cells, for example xenogeneic feeder cells, such as mouse embryonic fibroblasts. The cells and tissue according to the present disclosure do not contain any non-human components because they are from fully human origin. However, media employed to culture the cells may contain, for example foetal calf serum. This serum does not render the cells and tissue of the present disclosure non-human.

As used herein “fibroblasts” are understood to be naturally occurring fibroblasts or their precursor cells, for example adipose-derived stromal cells, more particularly fibroblasts occurring in the dermis, genetically modified fibroblasts or fibroblasts emanating from spontaneous mutations or precursors thereof.

As used herein “keratinocytes” are understood to be cells of the epidermis which form keratinizing plate epithelium, genetically modified keratinocytes or keratinocytes emanating from spontaneous mutations or precursors of such keratinocytes which may be of animal or human origin. Alternatively, to the normal skin keratinocytes, mucous membrane keratinocytes or intestinal epithelial cells may be applied to the matrix. These are, for example pre-cultivated cells and, in one embodiment, keratinocytes in the first or in the second cell passage, although cells from higher passages may also be used.

The fibroblasts and keratinocytes are obtained and cultivated by methods known among skilled addressees which may be adapted to the required properties of the skin tissue to be produced.

In one embodiment other cell types and/or other cells of other tissue types, for example, melanocytes, macrophages, monocytes, leukocytes, plasma cells, neuronal cells, adipocytes, induced and non-induced precursor cells of Langerhans cells, Langerhans cells and other immune cells, endothelial cells, more particularly sebocytes or sebaceous gland tissue or sebaceous gland explantates, cells of the sweat glands or sweat gland tissue or sweat gland explantates, hair follicle cells or hair follicle explantates; and cells from tumors of other organs or from metastases, may be cultured together with the human keratinocytes. The cells mentioned may be of human and animal origin but unless mentioned otherwise, will be human in order to produce a fully human epidermis. Stem cells of various origins, tissue-specific stem cells, embryonal and/or adult stem cells may also be incorporated in the skin model.

“Trypsin” as used herein (EC number 3.4.21.4) is a serine protease from the PA clan superfamily, found in the digestive system, such as in the pancreas of many vertebrates where it hydrolyses proteins. Trypsin cleaves peptide chains primarily at the carboxyl side of the amino acids lysine or arginine. The rate of hydrolysis is slower if an acidic residue is on either side of the cleavage site and no cleavage occurs if a proline residue is on the carboxyl side of the cleavage site. As used in the presently disclosed method, trypsin is able to digest both the epidermis and dermis layers of skin samples.

“Collagenase” as used herein refers to a group of enzymes which break down the native collagen that holds animal tissues together. Collagenases are made by a variety of different microorganisms and by many different animal cells. Crude collagenase preparations contain several isoforms of two different collagenases, a sulfhydryl protease, clostripain, a trypsin-like enzyme, and an aminopeptidase. This combination of collagenolytic and proteolytic activities is effective at breaking down intercellular matrices, the essential part of tissue dissociation. One component of the complex is a hydrolytic enzyme which degrades the helical regions in native collagen preferentially at the Y-Gly bond in the sequence Pro-Y-Gly-Pro, where Y is most frequently a neutral amino acid. This cleavage yields products susceptible to further peptidase digestion. Crude collagenase is inhibited by metal chelating agents such as cysteine, EDTA or o-phenanthroline but not DFP. It is also inhibited by a2-macroglobulin, a large plasma glycoprotein. Ca² is required for enzyme activity. 4 main types of collagenase are typically used depending on the requirements:
Type 1 crude collagenase has the original balance of collagenase, caseinase, clostripain and tryptic activities.
Type 2 contains higher relative levels of protease activity, particularly clostripain.
Type 3 contains lowest levels of secondary proteases.
Type 4 is designed to be especially low in tryptic activity to limit damage to membrane proteins and receptors.

In one embodiment, Type 1 collagenase employed in the methods of the present disclosure.

“Feeder cells” as used herein refers to a population of cells, typically connective tissue cells that are used to nourish cultured tissue cells, in particular the human keratinocytes as described herein. The feeder cells supply metabolites and other nutrients to the cells they support. Feeder cells typically do not grow or divide and are usually inactivated by irradiation, for example gamma irradiation. However, in the present disclosure the feeder cells are unirradiated.

In one embodiment, the human fibroblast cells function as feeder cells to support the growth of the human keratinocytes. In one embodiment, the human fibroblast cells are not irradiated. This means that the human fibroblast cells are not inactivated and are able to continue growing in tandem with the human keratinocytes in the culture. This is possible because the presence of the ROCK inhibitor enables the keratinocytes to grow at a rate that avoid being outgrown by the fibroblasts.

In one embodiment, the human fibroblast cells are matched to the human keratinocytes. For example, the fibroblasts may be sex matched and/or HLA matched to the keratinocytes.

In another embodiment, the human fibroblasts and human keratinocytes may both be derived from the same donor. Alternatively, the human keratinocytes and/or human fibroblast cells to be cultured are autologous, that is derived from the patient for whom the fully human epidermis is intended. As discussed above autologous/allogeneic is not technically relevant to performing the actual process steps, but is relevant to administration to a patient.

In one embodiment, the sowing of the skin cells on the matrix takes place in the presence of a physiological solution.

The term “physiological solution” as used herein refers to a solution that is similar or identical to one or more physiological condition or that can change the physiological state of a certain physiological environment. The term “physiological solution” as used herein also refers to a solution that is capable of supporting growth of cells (including, but not limited to, mammalian, vertebrate, and/or other cells).

In one embodiment, a physiological solution comprises a defined culture medium, in which the concentration of each of the medium components is known and/or controlled. Defined media typically contain all the nutrients necessary to support cell growth, including, but not limited to, salts, amino acid, vitamins, lipids, trace elements, and energy sources such a carbohydrates. Non-limiting examples of defined media include DMEM, Basal Media Eagle (BME), Medium 199; F-12 (Ham) Nutrient Mixture; F-IO (Ham) Nutrient Mixture; Minimal Essential Media (MEM), Williams' Media E, and RPMI 1640.

In one embodiment, the physiological solution is a cell culture medium as disclosed herein.

In one embodiment, the media may contain other factors, for example, hormones, growth factors, adhesion proteins, antibiotics, selection factors, enzymes and enzyme inhibitors and the like. Growth factors for example may help to enhance the proliferation of the seeded cells.

Antibody as employed herein refers to a full-length antibody, a binding fragment thereof, or an antibody molecule comprising any one of the same. Examples of antibody binding fragments include Fab, modified Fab, Fab′, modified Fab′, F(ab′)2, Fv, Fab-Fv, Fab-dsFv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217).

A peptide as employed herein is a sequence of 2 to 50 amino acids.

A synthetic peptide as employed herein refers to a peptide prepared by synthetic chemistry techniques (as opposed to peptides expressed recombinantly).

Substantially planar as employed herein refers to having a surface substantially (a major portion of which is) lying in one plane.

Substrates/Matrices of the Present Disclosure

The term “matrix”, “cell matrix”, “cellular matrix”, “substrate” or “cell substrate” Is used interchangeably herein, unless the context indicates otherwise. It refers to any physical structure including but not limited to, a solid or semi-solid structure, such as a meshwork of fibres with pores suitable for providing:
mechanical or other support for the adherence and proliferation of cells or tissue, and
allowing migration of at least one cell types during the culturing process. for example for ex vivo skin tissue culture.

In contact with the gas permeable layer/membrane as employed herein refers to the relevant cells being on the membrane/layer or in the proximity of the membrane/layer, such that the growth and/or in particular differentiation of the cells can occur. Thus proximity will generally mean that there is nothing separating the gas permeable layer/membrane and the relevant cells (the space therebetween will be filled for example with culture media, buffer or CO₂, in particular cell culture media). In one embodiment the distance of the relevant cells to the gas permeable layer is 2 cm or less, such as 1 cm or less, in particular 0.5, 0.4, 0.3, 0.2, 0.1 or 0.05 cm. In one embodiment the outer of cells for differentiation rest on the gas permeable layer/membrane.

Generally, the matrix will be three dimensional, with a first 2D face and second face 2D (with a significant surface area) on which cells may deposited and a depth between the two faces giving the third dimension (corresponding to a cross-section of the final skin—somewhere in the region of a 100 μm as discussed above).

The matrices of the present disclosure may be constructed of natural or synthetic materials. A matrix may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

In one embodiment, the matrices comprise only synthetic materials. In another embodiment the matrix comprises a mixture of synthetic and natural materials.

In one embodiment, synthetic materials for making the matrix of the present invention are both biocompatible and biodegradable (e.g. subject to enzymatic and hydrolytic degradation), such as biodegradable polymers.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

The term “biodegradable” or “bioabsorbable” as used herein is intended to describe materials that exist for a limited time in a biological environment and degrade under physiological conditions to form a product that can be metabolized or excreted without damage to the subject. In certain embodiments, the product is metabolized or excreted without permanent damage to the subject.

In one embodiment, the matrix is completely resorbable by the body of a subject.

In one embodiment, a bioabsorbable matrix of the present disclosure may exist for days, weeks or months when placed in the context of a biological environment. For example, a bioabsorbable matrix may exist for 1, 2, 3, 4, 5, 6, 7, 8, 9 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180 days or more when placed in the context of biological environment.

In one embodiment, the matrix layer is resorbed by the body of said subject at about a same rate as growth of tissue cells underlying said membrane matrix layer in said area. In certain embodiments, the cells are epithelial cells. In certain embodiments, the matrix layer is substantially completely resorbed by said body within about 3 to 12 months after the skin graft is applied. In certain embodiments, the matrix is substantially completely resorbed within about 3 months.

Biodegradable materials such as polymers may be hydrolytically degradable, may require cellular and/or enzymatic action to fully degrade, for example hydrolysis, oxidation, enzymatic processes, phagocytosis, or other processes, including a combination of the foregoing.

Biodegradable polymers are known to those of ordinary skill in the art and include, but are not limited to, synthetic polymers, natural polymers, blends of synthetic and natural polymers, inorganic materials, and the like.

In one embodiment, the matrix incorporates one or more synthetic polymers in its construction. The matrix may be made from heteropolymers, monopolymers, or combinations thereof. Examples of polymers suitable for manufacturing cell matrices include, but are not limited to aliphatic polyesters, copoly(ether-esters), polyalkylenes oxalates, polyamides, poly(iminocarbonates), polyorthoesters, polyoxaesters, polyamidoesters, polyoxaesters containing amine groups, poly(anhydrides), polyphosphazenes, biomolecules and blends thereof.

Suitable aliphatic polyesters include homopolymers, copolymers (random, block, segmented, tappered blocks, graft, triblock, etc.) having a linear, branched or star structure. Suitable monomers for making aliphatic homopolymers and copolymers may be selected from the group consisting of, but are not limited, to lactic acid, lactide (including L-, D-, meso and D,L mixtures), glycolic acid, glycolide, epsilon-caprolactone, p-dioxanone (1,4-dioxan-2-one), trimethylene carbonate (1,3-dioxan-2-one), delta-valerolactone, beta-butyrolactone, epsilon-decalactone, 2,5-diketomorpholine pivalolactone, alpha, alpha-diethylpropiolactone, ethylene carbonate, ethylene oxalate, 3-methyl-1,4-dioxane-2,5-dione, 3,3-diethyl-1,4-dioxan-2,5-dione, gamma-butyrolactone, 1,4-dioxepan-2-one, 1,5-dioxepan-2-one, 6,6-dimethyl-dioxepan-2-one, 6,8-dioxabicycloctane-7-one and combinations thereof. In one embodiment the synthetic polymers are selected from PLGA, PLA, PCL, PHBV, PDO, PGA, PLCL, PLLA-DLA, PEUU, cellulose-acetate, PEG-b-PLA, EVOH, PVA, PEO, PVP, blended PLA/PCL, PDLA/HA, PLLA/HA, PLLA/MWNTs/HA, PLGA/HA, 100 dioxanone linear homopolyer and combinations of two or more of the same.

Elastomeric copolymers also are particularly useful in the presently disclosed matrices. Suitable bioabsorbable biocompatible elastomers include but are not limited to those selected from the group consisting of elastomeric copolymers of epsilon-caprolactone and glycolide (suitably having a mole ratio of epsilon-caprolactone to glycolide from about 35:65 to about 65:35, for example from 45:55 to 35:65) elastomeric copolymers of epsilon-caprolactone and lactide, including L-lactide, D-lactide blends thereof or tactic acid copolymers (suitably having a mole ratio of epsiton-caprolactone to lactide of from about 35:65 to about 65:35 and, for example from 45:55 to 30:70 or from about 95:5 to about 85:15) elastomeric copolymers of p-dioxanone (1,4-dioxan-2-one) and lactide including L-lactide, D-lactide and lactic acid (preferably having a mole ratio of p-dioxanone to lactide of from about 40:60 to about 60:40) elastomeric copolymers of epsilon-caprolactone and p-dioxanone (suitably having a mole ratio of epsilon-caprolactone to p-dioxanone of from about from 30:70 to about 70:30) elastomeric copolymers of p-dioxanone and trimethylene carbonate (suitably having a mole ratio of p-dioxanone to trimethylene carbonate of from about 30:70 to about 70:30), elastomeric copolymers of trimethylene carbonate and glycolide (suitably having a mole ratio of trimethylene carbonate to glycolide of from about 30:70 to about 70:30), elastomeric copolymer of trimethylene carbonate and lactide including L-lactide, D-lactide, blends thereof or lactic acid copolymers (suitably having a mole ratio of trimethylene carbonate to lactide of from about 30:70 to about 70:30) and blends thereof. Examples of suitable bioabsorbable elastomers are described in U.S. Pat. Nos. 4,045,418; 4,057,537 and 5,468,253 all hereby incorporated by reference. These elastomeric polymers will have an inherent viscosity of from about 1.2 dL/g to about 4 dL/g, preferably an inherent viscosity of from about 1.2 dL/g to about 2 dL/g and, for example an inherent viscosity of from about 1.4 dL/g to about 2 dL/g as determined at 25° C. in a 0.1 gram per deciliter (g/dL) solution of polymer in hexafluoroisopropanol (HFIP).

Other materials suitable for use as a matrix of the present disclosure include, but are not limited to, polylactic acid-glycolic acid (PLGA), polyorthoesters, polyanhydrides, polyphosphazenes, and combinations thereof. Non-biodegradable polymers include polyacrylates, polymethacrylates, ethylene vinyl acetate, polyvinyl alcohols, polylactide, chondroitin sulfate (a proteoglycan component), polyesters, polyethylene glycols, polycarbonates, polyvinyl alcohols, polyacrylamides, polyamides, polyacrylates, polyesters, polyetheresters, polymethacrylates, polyurethanes, polycaprotactone, polyphophazenes, polyorthoesters, polyglycolide, copolymers of lysine and lactic acid, copolymers of lysine-RGD and lactic acid, and the like, and copolymers of the same. Synthetic polymers can further include those selected from the group consisting of aliphatic polyesters, poly(amino acids), poly(propylene fumarate), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, and blends thereof.

In one embodiment, the matrix incorporates polylactic acid (PLA). PLA is particularly suited to tissue engineering methods using the cellular matrix as PLA degrades within the human body to form lactic acid, a naturally occurring chemical which is easily removed from the body. The cellular matrix of the invention may also incorporate polyglycolic acid (PGA) and/or polycaprolactone (PCL) as matrix materials. PGA and PCL have similar degradation pathways to PLA, but PGA degrades in the body more quickly than PLA, while PCL has a slower degradation rate than PLA.

PGA has been widely used in tissue engineering. PGA matrices can be easily manipulated into various three dimensional structures, and offer an excellent means of support and transportation for cells (Christenson L, Mikos A G, Gibbons D F, et al: Biomaterials for tissue engineering: summary. Tissue Eng. 3 (1): 71-73; discussion 73-76, 1997). Matrices manufactured from polyglycolic acid alone, as well as combinations of PGA and other natural and/or synthetic biocompatible materials, are within the scope of the present disclosure.

In one embodiment, the matrix comprises poly(lactic-co-glycolic acid) (PLGA), such as PLGA microfiber or nanofibres.

In another embodiment, the matrix comprises dioxanone linear homopolymer, such as 100 dioxanone linear homopolymer (e.g. Dioxaprene 100M).

In one embodiment, the matrix comprises a combination of PLGA and 100 Dioxanone.

The term “fibre” is used herein to refer to materials that are in the form of continuous filaments or discrete elongated pieces of material, typically comprising or composed of biodegradeable polymers such as those described above. The fibres of the present disclosure typically have diameters in the micrometer range, such as 0.5 μm to 5 μm, for example 1 μm, 1.5 μm, 2 μm, 2.5 μm, 3 μm, 3.5 μm, 4 μm, 4.5 μm or 5 μm, in particular in the range 1 to 3 μm.

The term “fibre matrix” is used herein to refer to the arrangement of fibres into a supporting framework, such as in the form of a sheet of fibres that can then be used to support cells or other additional materials (see also definition of “matrix” above). Various methods are known to the skilled person which can be used to produce suitable fibers, include, but are not limited to, interfacial polymerization and electrospinning.

In one embodiment, a matrix of the present disclosure is formed using electrospinning.

The term “electrospinning” generally refers to techniques that make use of a high-voltage power supply, a spinneret (e.g., a hypodermic needle), and an electrically conductive collector plate (e.g., aluminum foil or stainless steel). To perform the electrospinning process using these materials, an electrospinning liquid (i.e. a melt or solution of the desired materials that will be used to form the fibers) is generally first loaded into a syringe and is then fed at a specific rate set by a syringe pump.

As the liquid is fed by the syringe pump with a sufficiently high voltage, the repulsion between the charges immobilized on the surface of the resulting liquid droplet overcomes the confinement of surface tension and induces the ejection of a liquid jet from the orifice. The charged jet then goes through a whipping and stretching process, and subsequently results in the formation of uniform nanofibers. Further, as the jet is stretched and the solvent is evaporated, the diameters of the fibres can then be continuously reduced to a desired scale, for example micrometers, or even as small as nanometers and, under the influence of an electrical field, the fibres can subsequently be forced to travel towards a grounded collector, onto which they are typically deposited as a non-woven mat. In the context of the present disclosure, due to the high ratio of surface area to volume and the one-dimensional morphology, electrospun fibres can mimic the architecture of the extracellular matrix.

Examples of materials used to produce the nanofibers of the present disclosure are selected from those listed in Tables 1 and 2 below.

TABLE 1
Exemplary Materials for producing electrospun fibres
(natural polymers).

| Materials | Solvent | Materials | Solvent |
|---|---|---|---|
| Chitosan | 90% Acetic | Virus M13 | THF |
| | acid | viruses | |
| Gelatin | Formic | Hemoglobin | TFE |
| | acid | | |
| Gelatin | TFE | Phospholipids | Chloroform/ |
| | | (Lecithin) | DMF |
| Collagen Type I, | HFIP | Hyaluronic acid | DMF/water |
| II, & III | | | |
| Collagen Type I, | HFIP | Fibrinogen | HFIP/10 × |
| II, and III | | | minimal |
| | | | essential medium |
| Collagen Type I, | HFIP | Fibrous calf thymus | Water/ethanol |
| II, and III | | Na-DNA | |
| Elastin | HFIP | Silk fibroin | Methanol |
| Cellulose | NMMO/ | | |
| | water | | |

TABLE 2
Exemplary Materials for producing electrospun
fibres (synthetic polymers).

| Materials | Solvent | Materials | Solvent |
|---|---|---|---|
| PLGA | THF/DMF | PLA | HFIP |
| PLA | DCM | PLA | DCM/DMF |
| PLA | DCM/pyridine | PCL | DCM/ |
| | | | methanol |
| PHBV | Chloroform/ | PDO | HFIP |
| | DMF | | |
| PGA | HFIP | PLCL | Acetone |
| PLCL | DCM | PLLA-DLA | Chloroform |
| PEUU | HFIP | Cellulose acetate | Acetic acid/ |
| | | | water |
| PEG-b-PLA | Chloroform | PVA | Water |
| Collagen/ | TFE/water | EVOH | 70% propan- |
| chondroitin | | | 2-ol/water |
| sulfate | | | |
| PEO | Water | PVP | Ethanol/ |
| | | | water |

Blended

| | | | |
|---|---|---|---|
| PCL/collagen | HFIP | Sodium | Water |
| | | aliginate/PEO | |
| Chitosan/PEO | Acetic acid/ | Chitosan/PVA | Acetic acid |
| | DMSO | | |
| Gelatin/elastin/ | HFIP | Silk/PEO | Water |
| PLGA | | | |
| Silk fibroin/ | Formic acid | PDO/elastin | HFIP |
| chitosan | | | |
| PHBV/collagen | HFIP | Hyaluronic | DMF/water |
| | | acid/gelatin | |
| Collagen/chitosan | HFIP/TFA | | |

Composites

| | | | |
|---|---|---|---|
| PDLA/HA | Chloroform | PCL/CaCO3 | Chloroform/ |
| | | | methanol |
| PCL/CaCO3 | DCM/DMF | PCL/HA | DCM/DMF |
| PLLA/HA | Chloroform | Gelatin/HA | HFIP |
| PCL/collagen/HA | HFIP | Collagen/HA | HFIP |
| Gelatin/siloxane | Acetic acid/ethyl | PLLA/MWNTs/ | 1,4-dioxane/ |
| | acetate/water | HA | DCM |
| PLGA/HA | DCM/water | | |

In one embodiment, the matrix of the present disclosure is composed of synthetic microfibers or nanofibres, for example using the materials listed in Table 2.

The selection of a particular polymer and its use in a specified amount or concentration, or range thereof, provides the ability to control, customize and tailor the degradation rate of the polymer and therefore, the degradation rate of the matrix. This is useful because it is desirable for the matrix to remain as part of the skin graft in order to provide structural support to the grown skin tissue but to eventually degrade and be bioabsorbed by the patient's body once the patient's own cells have assimilated the skin graft, thereby eliminating the requirement for the matrix to be retrieved from the patient's body later on.

Various blends of polymers, for example made by electrospinning using the materials listed in Tables 1 and 2, may be used to form the fibres to improve their biocompatibility as well as their mechanical, physical, and chemical properties.

Once the desired microfiber or nanofiber matrices have been produced, in one embodiment two or more fibre matrices of the present disclosure are layered together. By layering multiple fibre matrices, the advantages of each fibre matrix can be combined, and in some cases, result in a synergistic effect.

For example, a first matrix may comprise microwells for receiving one or more relevant cells and/or skin tissue, which is then layered on a second matrix having radially-aligned fibres. In this example, the first matrix can provide the benefit of increasing the repair of damaged skin by providing relevant cells and/or skin tissue whereas the second matrix can provide the benefit of directing and enhancing cell migration from the periphery to the centre of the layered matrices. Layering two or more matrices may also help to enhance the watertight properties of a matrix. The skilled person is able to derive various combinations of two or more different matrices in order to achieve desired properties.

In one embodiment, the matrix of the present disclosure may be further treated via a single procedure or a combination of procedures which reduce the number of microorganisms capable of growing in the matrix under conditions at which the matrix is stored and/or distributed.

In one embodiment, the matrix is sterilised using gamma radiation. In another embodiment, the matrix is sterilised using ethylene oxide (EtO). In another embodiment, the matrix is sterilised using Revox which utilises percetic acid.

In one embodiment, the matrix is sterilized using ionizing radiation such as E-beam irradiation. Electron beam processing has the shortest process cycle of any currently recognized sterilization method. E-beam irradiation, products are exposed to radiation for seconds, with the bulk of the processing time consumed in transporting products into and out of the radiation shielding. Overall process time, including transport time, is 5 to 7 minutes. Electron beam processing involves the use of high energy electrons, typically with energies ranging from 3 to 10 million electron volts (MeV), for the radiation of single use disposable medical products. The electrons are generated by accelerators that operate in both a pulse and continuous beam mode. These high energy levels are required to penetrate product that is, for example packaged in its final shipping container. As the beam is scanned through the product, the electrons interact with materials and create secondary energetic species, such as electrons, ion pairs, and free radicals. These secondary energetic species are responsible for the inactivation of the microorganisms as they disrupt the DNA chain of the microorganism, thus rendering the product sterile. The skilled addressee is aware of other possible methods for sterilising the matrices of the present disclosure.

The seeding densities of the cellular matrix may vary and the individual layers of the cell matrix may have the same or different seeding densities. Seeding densities may vary according to the particular application for which the cellular matrix is applied. Seeding densities may also vary according to the cell type that is used in manufacturing the cellular matrix.

The number and concentration of cells seeded into or onto the matrix can be varied by modifying the concentration of cells in suspension, or by modifying the quantity of suspension that is distributed onto a given area or volume of the matrix.

In one embodiment, the seeding density is about 150,000 keratinocytes/cm² or higher such as 200,000, 250,000, 300,000, 350,000, 400,000, 450,000, 500,000, 550,000 or 600,000 keratinocytes/cm².

In one embodiment, the seeding density is about 50,000 fibroblasts/cm² or higher, such as 60,000, 70,000, 80,000, 90,000, 100,000, 110,000, 120,000, 130,000, 140,000, 150,000, 160,000, 170,000, 180,000, 190,000 or 200,000 fibroblasts/cm².

Seeding densities of the individual layers of the matrix will depend on the use for which the matrix is intended. Although one skilled in the art may appreciate particular seeding densities a specific application will require, individual layers of the matrix may be seeded at a variety of seeding densities. One skilled in the art will appreciate that the seeding densities for the individual layers of the matrix may vary according to the use for which the matrix is intended.

Spreading involves the use of an instrument, such as a spatula, to spread the inoculum across the spongiform matrix. Seeding the matrix by painting is accomplished by dipping a brush into the inoculum, withdrawing it, and wiping the inoculum-laden brush across the matrix. This method suffers the disadvantage that substantial numbers of cells may cling to the brush, and not be applied to the lattice. However, it may nevertheless be useful, especially in situations where it is desired to carefully control the pattern or area of lattice over which the inoculum is distributed.

Seeding the matrix by spraying generally involves forcing the inoculum through any type of nozzle that transforms liquid into small airborne droplets. This embodiment is subject to two constraints. First, it must not subject the cells in solution to shearing forces or pressures that would damage or kill substantial numbers of cells. Second, it should not require that the cellular suspension be mixed with a propellant fluid that is toxic or detrimental to cells or wound beds. A variety of nozzles that are commonly available satisfy both constraints. Such nozzles may be connected in any conventional way to a reservoir that contains an inoculum of epithelial stem cells.

Seeding the matrix by pipetting is accomplished using pipettes, common “eye-droppers,” or other similar devices capable of placing small quantities of the inoculum on the surface of the matrix of the present disclosure. The aqueous liquid will permeate through the porous matrix. The cells in suspension tend to become enmeshed at the surface of the matrix and are thereby retained upon the matrix surface.

According to another embodiment of the invention, an inoculum of cells may be seeded by means of a hypodermic syringe equipped with a hollow needle or other conduit. A suspension of cells is administered into the cylinder of the syringe, and the needle is inserted into the matrix. The plunger of the syringe is depressed to eject a quantity of solution out of the cylinder, through the needle, and into the scaffold.

An important advantage of utilizing an aqueous suspension of cells is that it can be used to greatly expand the area of matrix on which an effective inoculum is distributed. This provides two distinct advantages. First, if a very limited amount of intact tissue is available for autografting, then the various suspension methods may be used to dramatically increase the area or volume of a matrix that may be seeded with the limited number of available cells. Second, if a given area or volume of a matrix needs to be seeded with cells, then the amount of intact tissue that needs to be harvested from a donor site may be greatly reduced. The optimal seeding densities for specific applications may be determined through routine experimentation by persons skilled in the art.

Typically, the dimensions of the matrix should be substantially planar and of a thickness that gives seeded cells sufficient access to a nutrient medium. When implanted, the cell matrix must have sufficient access to body fluids for nutrition and waste removal. The thickness of the matrix may be varied by changes in the matrix's porosity. Thus, increases in matrix porosity may permit matrices to take on greater thickness as larger pore sizes improve access to external medium and body fluids.

Accordingly, in one embodiment, the matrix has a thickness of 100 μm or less, for example 100, 90, 80, 70, 60, 50, 40, 30, 20, 10, or 5 μm. By keeping the matrix 100 μm or less in thickness, this allows the seeded keratinocytes to receive nutrients and remove waste by diffusion alone, without requiring a vasculature system in order to survive.

Seeding the layered matrix involves introducing one or more desired cell populations to a selected substrate material and, for example subsequently joining the materials to create a layered matrix. Alternatively, the matrices may be pre-joined, and the selected population(s) of cells introduced at a selected location. Seeding is distinct from the spontaneous infiltration and migration of cells into the matrix from, for example a wound site when the matrix is placed at the wound site.

In one embodiment the matrices are seeded on at least one surface before the respective cell-seeded surfaces are opposed to each other to form a layered arrangement.

Various additional materials and/or biological molecules can be attached to the matrices of the present disclosure. The term “attached” includes, but is not limited to, coating, embedding or incorporating by any means the additional materials and/or biological molecules, and attached can refer to incorporating such components on the entire matrix or only a portion thereof.

In one embodiment, cell factors are coated/attached to the matrix of the present disclosure. As used herein, the term “cell factors” refers to substances that are synthesized by living cells (e.g. stem cells) and which produce a beneficial effect in the body (e.g. mammalian or human body). Cell factors include, but are in no way limited to, growth factors, regulatory factors, hormones, enzymes, lymphokines, peptides and combinations thereof. Cell factors may have varying effects including, but not limited to, influencing the growth, proliferation, commitment, and/or differentiation of cells (e.g. stem cells) either in vivo and/or in vitro.

Some non-limiting examples of cell factors include, but are not limited to, cytokines (e.g. common beta chain, common gamma chain, and IL-6 cytokine families), vascular endothelial growth factor (e.g. VEGF-A, -B, -C, -D, and -E), adrenomedullin, insulin-like growth factor, epidermal growth factor EGF, fibroblast growth factor FGF, autocrin motility factor, GDF, IGF, PDGF, growth differentiation factor 9, erythropoietin, activins, TGF-α, TGF-β, bone morphogenetic proteins (BMPs), Hedgehog molecules, Wnt-related molecules, and combinations thereof.

In one embodiment, a growth factor such as EGF (Epidermal Growth Factor), IGF-I (Insulin-like Growth Factor), a member of Fibroblast Growth Factor family (FGF), Keratinocyte Growth Factor (KGF), PDGF (Platelet-derived Growth Factor AA, AB, BB), TGF-β (Transforming Growth Factor family—β1, β2, β3), CIF (Cartilage Inducing Factor), at least one of BMP's 1-14 (Bone Morphogenic Proteins), Granulocyte-macrophage colony-stimulating factor (GM-CSF), or combinations thereof, which may promote tissue regeneration, can be attached to or coated to the matrices of the present disclosure.

In one embodiment, the growth factor is VEGF. In another embodiment, the growth factor is PDGF. The skilled addressee is aware of various other materials and biological molecules which may be attached to or used to coat a matrix of the presently-disclosed subject matter, and can be selected for a particular application based on the tissue to which they are to be applied.

In one embodiment, an extracellular matrix protein, such as, fibronectin, laminin, and/or collagen, is further attached to or coated on the matrix. Thus, in one embodiment, the matrix is coated with collagen IV, collagen I, laminin and fibronectin, or a combination thereof. The present inventors have discovered that these proteins help provide a secondary cellular signal which in conjunction with growth at an air liquid interface (such as on a gas permeable membrane), causes proper stratification of skin cells grown using the matrix.

In one embodiment, collagen IV is used. Collagen IV was shown to be particularly effective at producing proper epidermal stratification.

The extracellular matrix proteins may be in the form of full length proteins or peptides thereof, for example synthetic peptides.

In another embodiment, a therapeutic agent is further attached to the matrix. The term “therapeutic agent” as used herein refers to any of a variety of agents that exhibit one or more beneficial therapeutic effects when used in conjunction with methods, matrices and/or skin tissues of the present disclosure. Examples of therapeutic agents that may be used include, without limitation, proteins, peptides, drugs, cytokines, extracellular matrix molecules, and/or growth factors. One of skill in the art will be aware of other suitable and/or advantageous therapeutic agents that may be used in accordance with the present disclosure.

In one embodiment, the therapeutic agent is an anti-inflammatory agent or an antibiotic. Examples of anti-inflammatory agents that can be incorporated into the matrices include, but are not limited to, steroidal anti-inflammatory agents such as betamethasone, triamcinolone dexamethasone, prednisone, mometasone, fluticasone, beclomethasone, flunisolide, and budesonide; and non-steroidal anti-inflammatory agents, such as fenoprofen, flurbiprofen, ibuprofen, ketoprofen, naproxen, oxaprozin, diclofenac, etodolac, indomethacin, ketorolac, nabumetone, sulindac tolmetin meclofenamate, mefenamic acid, piroxicam, and suprofen.

Various antibiotics can also be employed in accordance with the presently-disclosed subject matter. Non-limiting examples include aminoglycosides, such as amikacin, gentamicin, kanamycin, neomycin, netilmicin, paromomycin, streptomycin, or tobramycin; carbapenems, such as ertapenem, imipenem, meropenem; chloramphenicol; fluoroquinolones, such as ciprofloxacin, gatifloxacin, gemifloxacin, grepafloxacin, levofloxacin, lomefloxacin, moxifloxacin, norfloxacin, ofloxacin, sparfloxacin, or trovafloxacin; glycopeptides, such as vancomycin; lincosamides, such as clindamycin; macrolides/ketolides, such as azithromycin, clarithromycin, dirithromycin, erythromycin, or telithromycin; cephalosporins, such as cefadroxil, cefazolin, cephalexin, cephalothin, cephapirin, cephradine, cefaclor, cefamandole, cefonicid, cefotetan, cefoxitin, cefprozil, cefuroxime, loracarbef, cefdinir, cefditoren, cefixime, cefoperazone, cefotaxime, cefpodoxime, ceftazidime, ceftibuten, ceftizoxime, ceftriaxone, or cefepime; monobactams, such as aztreonam; nitroimidazoles, such as metronidazole; oxazolidinones, such as linezolid; penicillins, such as amoxicillin, amoxicillin/clavulanate, ampicillin, ampicillin/sulbactam, bacampicillin, carbenicillin, cloxacillin, dicloxacillin, methicillin, mezlocillin, nafcillin, oxacillin, penicillin G, penicillin V, piperacillin, piperacillin/tazobactam, ticarcillin, or ticarcillin/clavulanate; streptogramins, such as quinupristin/dalfopristin; sulfonamide/folate antagonists, such as sulfamethoxazole/trimethoprim; tetracyclines, such as demeclocycline, doxycycline, minocycline, or tetracycline; azole antifungals, such as clotrimazole, fluconazole, itraconazole, ketoconazole, miconazole, or voriconazole; polyene antifungals, such as amphotericin B or nystatin; echinocandin antifungals, such as caspofungin or micafungin, or other antifungals, such as ciclopirox, flucytosine, griseofulvin, or terbinafine.

In one embodiment, various analgesic and/or anesthetic are attached to or incorporated into the matrices of the presently disclosure. As used herein, the term “analgesic” refers to agents used to relieve pain and, in some embodiments, can be used interchangeably with the term “anti-inflammatory agent” such that the term analgesics can be inclusive of the exemplary anti-inflammatory agents described herein. Exemplary analgesic include, but are not limited to: paracetamol and non-steroidal anti-inflammatory agents, COX-2 inhibitors, and opiates, such as morphine, and morphinomimetics.

As used herein, the term “anesthetic” refers to agents used to cause a reversible loss of sensation in subject and can thereby be used to relieve pain. Exemplary anesthetics that can be used in accordance with the presently-disclosed subject matter include, but are not limited to, local anesthetics, such as procaine, amethocaine, cocaine, lidocaine, prilocaine, bupivicaine, levobupivicaine, ropivacaine, mepivacaine, and dibucaine.

Culture Devices of the Present Disclosure

The methods of the present disclosure may be carried out using any cell culture device suitable for the production of fully human epidermis. WO2016/209089, the contents of which are incorporated by reference, describes such devices. The skilled person will be aware of other alternative culture devices.

Uses of the Fully Human Epidermis of the Present Disclosure

The present disclosure provides for the use of tissue, such as epithelium, epidermis, stratified epithelium, stratified epidermis and dermis, split thickness skin or full thickness skin, prepared using a method described herein, for example using a matrix of the present disclosure, for the treatment of tissue damage in subject in need thereof.

The term “subject” is used herein to refer to both human and animal subjects but is generally intended to refer to a human patient in need of treatment.

The terms “treatment” or “treating,” as used herein include, but are not limited to, inhibiting the progression of damage to a tissue, arresting the development of damage to a tissue, reducing the severity of damage to a tissue, ameliorating or relieving symptoms associated with damage to a tissue, and repairing, regenerating, and/or causing a regression of damaged tissue or one or more of the symptoms associated with a damaged tissue.

The present disclosure provides to a method of treating tissue damage in a subject in need thereof comprising:
a) providing a skin tissue, such as epithelium, stratified epithelium, epidermis, stratified epidermis, stratified epidermis and dermis, split thickness skin or full-thickness skin, which has been grown, for example in a method as herein described, such as a method employing a fully human epidermis produced using the method described herein,
b) recovering under sterile or aseptic conditions the tissue with the matrix, and
c) applying the tissue to the patient.

In various embodiments the tissue damage is a wound, a chronic wound, a surgical wound, an ulcer, a non-healing wound, a scar, a surgical scar, a scald or a burn. In various embodiments the burn is a first degree burn, a second degree burn, a third degree burn, a deep dermal burn or a full thickness burn.

In various embodiments the tissue damage is epithelium located on a mucosal surface. In various embodiments the epithelium is located on or in skin, the lungs, the gastrointestinal tract (for example, the oesophagus or mouth), reproductive tract, or the urinary tract (for example, the urethra).

Other examples of treatments include but are not limited to: skin regeneration with nerves & organelles; wound healing, for example promoting/enhancing wound healing, including ulcers such as diabetic ulcers; burn healing; skin regeneration and repair; epidermolysis bullosa; enhance skin quality or appearance; prevention or remediation of skin disorders; diminishment or abolishment of scar tissues; breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring.

In one embodiment, the present disclosure provides a matrix, skin tissue and method useful in the regeneration of damaged, lost and/or degenerated tissue. For example, a matrix, method or skin tissue of the present invention may be employed to initiate, increase, support, promote, and/or direct the regeneration of damaged, lost, and/or degenerated tissue, in particular the regeneration of damaged skin.

“Regeneration”, “Regenerate”, “Regenerative” as used herein refer to any process or quality that initiates, increases, modulates, promotes, supports, and/or directs the growth, regrowth, repair, functionality, patterning, connectivity, strengthening, vitality, and/or the natural wound healing process of weak, damaged, lost, and/or degenerating tissue. These terms can also refer to any process or quality that initiates, increases, modulates, promotes, supports, and/or directs the growth, strengthening, functionality, vitality, toughness, potency, and/or health of weak, tired, and/or normal tissue.

As used herein, the term “wound” is used to refer broadly to injuries to the skin and subcutaneous tissue initiated in different ways (e.g., pressure sores from extended bed rest and wounds induced by trauma) and with varying characteristics. Wounds are generally classified into one of four grades depending on the depth of the wound: Grade I: wounds limited to the epithelium; Grade II: wounds extending into the dermis; Grade III: wounds extending into the subcutaneous tissue; and Grade IV (or full-thickness wounds), which are wounds in which bones are exposed (e.g., a bony pressure point such as the greater trochanter or the sacrum).

As used herein, the term “partial thickness wound” refers to wounds that encompass Grades I-III; e.g., burn wounds, pressure sores, venous stasis ulcers, and diabetic ulcers. As used herein, the term “deep wound” is used to describe to both Grade III and Grade IV wounds.

In one embodiment, there is provided is a skin tissue such as epithelium, epidermis, stratified epithelium, stratified epidermis and dermis, split thickness skin or full-thickness skin, prepared using a method described herein for facilitating a skin graft, by covering an area of damaged, injured, wounded, diseased, removed or missing skin tissue of a body of a subject.

As used herein, a “graft” refers to a cell, tissue or organ that is implanted into an individual, typically to replace, correct or otherwise overcome a defect. Thus, a “skin graft” is a skin tissue that may be implanted into an individual, for example sutured to the individual. A graft may further comprise a matrix of the present disclosure, for example wherein the matrix is integrated into the skin graft. The tissue or organ may consist of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following interchangeable terms: “allograft”, “allogeneic transplant”, “allogeneic implant”, “allogeneic graft” and “heterologous graft”. A “xenograft”, “xenogeneic transplant” or “xenogeneic implant” refers to a graft from one individual to another of a different species.

In one embodiment the tissue is prepared using cells that are autologous to the subject. For example, in various embodiments the tissue is prepared using fibroblasts, keratinocytes, or fibroblasts and keratinocytes that are autologous to the subject. In an alternative embodiment the tissue is prepared using cells that are heterologous to the subject. In a further embodiment the tissue is prepared using a combination of cells, wherein some of the cells are autologous to the subject and some of the cells are heterologous to the subject.

It will be appreciated that cells (for example autologous to the subject) may be isolated using any method known in the art. For example, cells (for example autologous cells) may be isolated from a skin sample or skin biopsy taken from the subject by digesting the sample tissue and separating fibroblasts and/or keratinocytes from the digested tissue.

In one embodiment the tissue is an autograft, for example, a skin autograft. In various embodiments the tissue is an epidermal autograft, a split thickness skin autograft or a full thickness skin autograft. In another embodiment the tissue is an allogeneic graft.

It will be appreciated that the application of tissue prepared using cells autologous to the patient, such as an autograft, is highly desirable to reduce or prevent immune rejection of the tissue and to reduce the requirement for ongoing immunotherapy or another ancillary treatments.

In one embodiment the tissue further comprises the matrix. In another embodiment the tissue is separated from the matrix before application to the patient.

Generally, the application of tissue to the patient will be by surgery. In one embodiment, recovery under sterile or aseptic conditions is during or immediately prior to surgery, for example in the surgical suite.

Generally, the application of tissue to the patient will be at or adjacent the site of tissue damage. In various embodiments the tissues is applied to at least partially cover the site of tissue damage or to completely cover the site of tissue damage.

In one embodiment the tissue is applied to temporarily cover the site of tissue damage. In an alternative embodiment the tissue is applied to permanently cover the site of tissue damage.

Other Non-Medical Uses

The efficacy and safety of topically applied pharmaceutical, nutraceutical or cosmetic products are typically tested using animal skin or live animals, human cadaver skin or synthetic human skin models.

Morphological differences between animal and human skin means that the excised animal skin or live animals for the testing of products is not optimal. Furthermore, there is considerable ethical concern about the use of live animals or animal skins for testing cosmetic products, including bans on such testing in some countries. For these reasons, there is a strong desire to identify alternatives to animal models for the testing of such products.

Inconsistent and highly variable results have been observed when human cadaver skin is used for product testing.

Accordingly, cells or tissues, such as skin tissue prepared using the device or methods described herein are useful for in vitro testing of pharmaceuticals, nutraceuticals and/or cosmetic products.

In various embodiments cells or tissue prepared using the device or methods described herein are used to test transdermal penetration of a compound, to test the permeation of a compound across the epidermis, dermis or basement membrane, to test the efficacy of an active ingredient for treating or preventing a condition, for example, a skin condition, or to test the toxicity of a compound.

More particularly, the skin tissue produced in accordance with the present disclosure is suitable for testing products, for example, for effectiveness, unwanted side effects, for example, irritation, toxicity and inflammation or allergenic effects, or the compatibility of substances. These substances may be substances intended for potential use as medicaments, for example as dermatics, or substances which are constituents of cosmetics or even consumer goods which come into contact with the skin, such as laundry detergents, etc.

The skin tissue of the present disclosure may also be used, for example, for studying the absorption, transport and/or penetration of substances. It is also suitable for studying other agents (physical quantities), such as light or heat, radioactivity, sound, electromagnetic radiation, electrical fields, for example, for studying phototoxicity, i.e. the damaging effect of light of different wavelengths on cell structures. The skin tissue may also be used for studying wound healing and is also suitable for studying the effects of gases, aerosols, smoke and dusts on cell structures or the metabolism or gene expression.

In various embodiments the cells or tissue are used to determine if a compound of interest is a skin irritant, for example, to determine if a compound of interest induces a skin rash, inflammation, or contact dermatitis.

The effects of substances or agents on human skin can be determined, for example, from the release of substances, for example, cytokines or mediators, by cells of the human or animal skin model system and the effects on gene expression, metabolism, proliferation, differentiation and reorganization of those cells. Using processes for quantifying cell damage, more particularly using a vital dye, such as a tetrazolium derivative, it is possible, for example, to detect cytotoxic effects on skin cells. The testing of substances or agents using the skin tissue may comprise both histological processes and also immunological and/or molecular-biological processes.

A “test agent” as used herein is any substance that is evaluated for its ability to diagnose, cure, mitigate, treat, or prevent disease in a subject, or is intended to alter the structure or function of the body of a subject. Test agents include, but are not limited to, chemical compounds, biologic agents, proteins, peptides, nucleic acids, lipids, polysaccharides, supplements, signals, diagnostic agents and immune modulators. Test agents may further include electromagnetic and/or mechanical forces.

In another embodiment, the skin tissue produced in accordance with the disclosure may be used as a model system for studying skin diseases and for the development of new treatments for skin diseases. For example, cells of patients with a certain genetic or acquired skin disease may be used to establish patient-specific skin model systems which may in turn be used to study and evaluate the effectiveness of certain therapies and/or medicaments.

In one embodiment, the skin tissue may be populated with microorganisms, more particularly pathogenic microorganisms. Population with pathogenic or parasitic microorganisms, including, in particular, human-pathogenic microorganisms.

“Microorganisms” as used herein generally refers to fungi, bacteria and viruses. The microorganisms are preferably selected from fungi or pathogenic and/or parasitic bacteria known to infect skin. These include but are not limited to species of the genus Candida albicans, Trichophyton mentagrophytes, Malassezia furfur and Staphylococcus aureus.

Using a correspondingly populated skin tissue, it is possible to study both the process of a microorganism population, more particularly the infection process, by the microorganism itself and the response of the skin to that population. In addition, the effect of substances applied before, during or after the population on the population itself or on the effects of the population on the skin tissue can be studied.

In various embodiments the cells comprise fibroblasts, keratinocytes or immune cells, or a combination of any two or more thereof. In one embodiment the cells comprise fibroblasts and keratinocytes. In various embodiments the tissue is selected from the group comprising epidermis, stratified epidermis and dermis, stratified epidermis and dermis, split thickness skin or full thickness skin.

In various embodiments the compound is a pharmaceutical compound, a cosmetic compound or a nutraceutical compound.

In various embodiments the compound for testing is applied to tissue alone or in an admixture with pharmaceutically or cosmetically acceptable carriers, excipients or diluents.

In various embodiments the compound for testing is applied topically to the tissue in the form of a sterile cream, gel, pour-on or spot-on formulation, suspension, lotion, ointment, dusting powder, a drench, spray, drug-incorporated dressing, shampoo, collar or skin patch.

The term “gas permeable material” or “gas permeable membrane” as used herein means a material or membrane through which gas exchange may occur.

“Comprising” in the context of the present specification is intended to mean “including”.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The background of the present specification contains technical information relevant to the disclosure herein and may be used as the basis for an amendment.

The present application claims priority from GB1715930.2 filed 30 Sep. 2018, incorporated herein by reference. The priority document may be employed is as the basis of making corrections to errors in the present application.

The invention will now be described with reference to the following examples, which are merely illustrative and should not in any way be construed as limiting the scope of the present invention.

1. (canceled)

2. A cholera toxin-free cell culture medium consisting of components: DMEM, Ham's F12, serum, one or more antibiotic(s) and/or antimycotic(s), keratinocyte growth factor (KGF) and a ROCK inhibitor.

3. The cell culture medium according to claim 2, wherein the DMEM is high glucose.

4. The cell culture medium according to claim 2, wherein the serum is mammalian.

5. The cell culture medium according to claim 2, wherein the serum is present at a concentration in the range 5 to 15%.

6. The cell culture medium according to claim 2, wherein the cell culture medium comprises at least one antibiotic.

7. The cell culture medium according to claim 6, wherein the antibiotic is independently selected from penicillin, streptomycin, ampicillin, amoxicillin, carbenicillin, cefotaxime, gentamicin, kanamycin, neomycin, polymyxin, blasticidin, geneticin, Hygromycin 1, Mycophenolic acid, puromycin, zeocin and combinations of two or more of the same.

8. The cell culture medium according to claim 6, wherein the antibiotic is selected from gentamicin, penicillin, or streptomycin.

9. The cell culture medium according to claim 2, wherein the cell culture medium comprises an antimycotic.

10. The cell culture medium according to claim 9, wherein the antimycotic is selected from amphotericin B, nystatin, actinomycin D, fosmidomycin, mycophenolic acid or combinations of two or more of the same.

11. The cell culture medium according to claim 10, wherein the antimycotic is amphotericin B.

12. The cell culture medium according to claim 2, wherein the ratio of DMEM:Ham's F12 is 2.5 to 3.5:1.5 to 0.5.

13. The cell culture medium according to claim 2, wherein the KGF has a concentration in the range 10 ng/ml to 30 ng/ml.

14. The cell culture medium according to claim 2, wherein the ROCK inhibitor is a small molecule ROCK inhibitor.

15. The cell culture medium according to claim 14, wherein the ROCK inhibitor is selected from the group consisting of: SB 77207713, Y-27632, Fasudil, Ripasudil, Y39983, Wf-536, SLx-2119, an azabenimidazole-aminofurazan, DE-104, H-1152, ROKa inhibitor, XD-4000, HMN-1152, 4-(1-aminoalkyl]-N-(4-pyridyl]cyclohexane-carboxamide, rhostatin, BA-210, BA-207, BA-215, BA-285, BA-1037, Ki-23095, VAS-012, RKI-1447, GSK429286A, Y-30141, HA-100, H-7, iso H-7, H-89, HA-1004, HA-1077, H-8, H-9, KN-62, GSK269962, quinazoline and combinations of two or more of the same.

16. The cell culture medium according to claim 15, wherein the ROCK inhibitor is SB 772077B, Y-27632, or a combination of both.

17. The cell culture medium according to claim 2, wherein the ROCK inhibitor concentration is in the range 0.1 to 100 μM.

18. The cell culture medium according to claim 2, wherein the serum is foetal bovine serum.

19. The cell culture medium according to claim 2, wherein the serum is present at a concentration of 10%.

20. The cell culture medium according to claim 11, wherein the amphotericin B is present at a concentration in the range of 0.500 to 0.750 μg/ml.

21. The cell culture medium according to claim 17, wherein the ROCK inhibitor concentration is in the range of 0.1 to 0.95 μM.
